SEQUENCE LISTING (10 sequences)

SEQ ID NO: 1 - protein, 6 aa, Artificial Sequence
  synthetic substrate
XSDEVX

SEQ ID NO: 2 - protein, 6 aa, Artificial Sequence
  synthetic substrate
XSDEVX

SEQ ID NO: 3 - protein, 5 aa, Artificial Sequence
  synthetic substrate
XDEVX

SEQ ID NO: 4 - protein, 4 aa, Artificial Sequence
  synthetic substrate
XETX

SEQ ID NO: 5 - protein, 4 aa, Artificial Sequence
  synthetic substrate
XVAX

SEQ ID NO: 6 - protein, 4 aa, Artificial Sequence
  synthetic substrate
XEHX

SEQ ID NO: 7 - protein, 6 aa, Artificial Sequence
  synthetic substrate
XSDEVX

SEQ ID NO: 8 - protein, 10 aa, Artificial Sequence
  synthetic substrate
XPEGIYGVLX

SEQ ID NO: 9 - protein, 10 aa, Artificial Sequence
  synthetic substrate
XPEGIYGVLX

SEQ ID NO: 10 - protein, 6 aa, Artificial Sequence
  synthetic substrate
WSDEVX

(19) United States
(12) Patent Application Publication
(10) Pub. No.: US 2011/0039289 A1
(43) Pub. Date: Feb. 17, 2011

(54) FLUOROGENIC PEPTIDES AND THEIR METHOD OF PRODUCTION

(76) Inventors: Pierre Graves, Cardiff (GB); John A. Smith, Cardiff (GB)

Correspondence Address: NIXON & VANDERHYE, PC, 901 NORTH GLEBE ROAD, 11TH FLOOR, ARLINGTON, VA, 22203, US

(21) Appl. No.: 12/922,649

(22) PCT Filed: Mar. 14, 2008
(86) PCT No.: PCT/GB08/00930

§ 371 Date: Sep. 14, 2010

(51) Int. Cl. C12Q 1/37; C07K 1/04; C07K 1/06; C07D 219/06; C07D 219/08; C07K 2/00; C09K 3/00

(52) U.S. Cl. 435/23; 530/345; 530/333; 530/331; 546/103; 530/300; 252/183.11

(57) ABSTRACT

A method of making a fluorogenic peptide with an acridone fluorophor is provided, the method comprising the steps of: (a) providing an acridone derivative, said acridone derivative having first and second reactive groups (or precursors thereof), (b) providing a solid phase support provided with reactive species for reacting with the first reactive group of the acridone derivative (c) causing the solid phase support and acridone derivative to react so that the acridone derivative is attached to the solid phase support (d) providing the peptide or reagents for the formation of the peptide, and (e) subsequent to step (c), causing the reaction of the second acridone reactive group with the peptide or one or more of the reagents for the formation of the peptide.

The present invention relates to fluorogenic peptides, and methods for making peptides, in particular peptides where the reporter is an acridone derivative.

Acridone and derivatives thereof (such as 2-aminoacridone) are known as labels for large molecules. For example, 2-aminoacridone has been used as a fluorogenic reporter for peptides and carbohydrates. It is used because it is a fluorescent moiety whose fluorescent properties depend on the peptides to which it is attached. The labelled molecules are produced by reacting the peptide or carbohydrate with the acridone moiety. This produces the product of interest, but many undesired side products.

The method of the present invention seeks to address one or more problems of the prior art methods.

In accordance with a first aspect of the present invention, there is provided a method of making a fluorogenic peptide with an acridone fluorophor, the method comprising the steps of:

(a) providing an acridone derivative, said acridone derivative having first and second reactive groups (or precursors thereof),

(b) providing a solid phase support provided with reactive species for reacting with the first reactive group of the acridone derivative

(c) causing the solid phase support and acridone derivative to react so that the acridone derivative is attached to the solid phase support

(d) providing the peptide or reagents for the formation of the peptide, and

(e) subsequent to step (c), causing the reaction of the second acridone reactive group with the peptide or one or more of the reagents for the formation of the peptide.

The term “peptide”, as used herein, encompasses molecules comprising amino acid chains of any length but preferably between four and ten amino acid residues, including full-length proteins, in which amino acid residues are linked by covalent peptide (—C(O)NH—) or thiopeptide (—C(S)NH—) bonds. The term “peptide” encompasses purified natural products, or products which may be produced partially or wholly using recombinant or synthetic techniques.

The term “peptide” may refer to a peptide, an aggregate of a peptide such as a dimer or other multimer, a fusion peptide, a peptide variant, or derivative thereof. The term also includes modifications of the peptide, for example, peptides modified by glycosylation, acetylation, phosphorylation, pegylation, ubiquitination, and so forth.

For the avoidance of doubt, it is stated herein that the peptide may solely comprise amino acid residues (apart from the acridone moiety, of course). Alternatively, the peptide may comprise moieties other than amino acid residues and the acridone moiety. For example, the peptide may comprise a chain of amino acids linked to the acridone moiety via a linking group, such as a hydrocarbon group, preferably an alkanediyl group (such as —C₂H₂ₙ—). The peptide may comprise two or more chains of amino acids, said chains being separated by non-amino acid moieties. The peptide may also comprise a protease cleavage recognition sequence such as an enterokinase or thrombin recognition sequence, or a self-splicing element such as an intein.

The term “amino acid” as used herein refers to an organic acid whose molecule contains both an amino (—NH₂) group and either a carboxyl group (COOH) or a thiocarboxyl group (CSSH, or COSH, or CSOH). The amino group and the carboxyl or thiocarboxyl group are typically coupled with an alkyanediykl, arenediyl or heterocyclic-containing moiety. The term “amino acid” includes both naturally-occurring amino acids as well as synthetic amino acids. A list of synthetic amino acids may be found in “The Peptides”, vol. 5, 1983, Academic Press, Chapter 6 by D. C. Roberts and F. Vellaccio. Examples of synthetic amino acids include nitro-phenylalanine and nitro-tyrosine. When the amino acids are alpha-amino acids, either the L-optical isomer or the D-optical isomer can be used, the L-isomers being preferred.

Steps (a) to (e) are not necessarily sequential steps; for example, steps (a) to (c) may be performed simultaneously. Furthermore, the steps do not have to be carried-out in the order given (save that step (e) must be performed after step (c)).

The method provides an effective way of controlling the synthesis of fluorogenic peptides. The fluorophor allows the structure of the peptide to be probed using spectroscopy. Additionally, the presence of the fluorophor enables the use of the peptides in essays (for example, in assays suitable for determining the activity of proteolytic enzymes). Changes in structure can result in a change in fluorescence properties such as: lifetime, intensity or emission wavelength. For example, certain amino acids, in particular, tyrosine, tryptophan, nitrotyrosine and nitrophenylalanine can quench the fluorescence of acridone moieties. This can be seen as a decrease in fluorescence lifetime as a function of distance between the acridone moiety and the quenching amino acid. Conversely, cleavage of the peptide between the reporter fluorophor and the quenching amino acid results in an increase in fluorescence lifetime and fluorescence intensity. The peptide does not need to be cleaved to produce changes in the fluorescence properties of the reporter fluorophor. For example, phosphorylation of a quenching tyrosine residue results in an increase in fluorescence lifetime and fluorescence intensity. It is preferred that the acridone derivative added in step (a) includes or comprises a precursor of the second reactive group. Such a precursor may comprise or include a protecting moiety. Such a protecting moiety may be removed, forming the second reactive group which is then able to react.

Suitable first and second reactive groups include, but are not limited to, sulphonate, sulphate, sulphonic acid, carboxyl, carboxylate, primary amine (amino or —NH₂) and mono-substituted amino (corresponding to a secondary amine).

It is preferred that the first reactive group comprises an acid group. Suitable acid groups include, but are not limited to, a carboxyl group. Alternatively, the acridone derivative provided in step (a) may include or comprise a precursor of an acid group (for example, an ester group).

It is preferred that the acridone derivative provided in step (a) comprises an amino group as the second reactive group or a precursor of an amino group. Such a precursor of an amino-group is a mono or di-substituted amino group. The substituent group may be a protecting moiety, such as those that are often used in peptide synthesis. Such protecting moieties are well-known to those skilled in the art and some of these are described in Biopolymers (Peptide Science), vol. 55, 123-139, (2000), John Wiley & Sons, Inc. Examples of such protecting moieties include carbonyl moieties (such as tert-butoxy carbonyl (BOC), benzoyloxycarbonyl, 2-(4-biphenyl)isopropoxycarbonyl (BPOC), fluorenylmethoxy carbonyl (Fmoc), alpha, alpha-dimethyl-3,5-dimethoxybenzyloxycarbonyl (Ddz), 2-(4-nitrophenylsulfonyl)ethoxycarbonyl (Nsc), sulphonyl moieties (such as triphenylmethyl sulphonyl moieties (such as mono- or di-nitro benzenesulphonyl), dithiasuccinoyl moieties, sulphonamide moieties, 1-(4,4-dimethyl-2,6-dioxocyclohexylidene)ethyl (Dde) moiety, aralkyl moieties (such as trityl), aryl moieties (optionally substituted, such as dimethoxybenzyl), diphenylmethyleneamine group, and phthalimide moieties.

One or both of the first and second reactive groups (or precursors thereof) may be linked to the acridone ring by a spacer group. The spacer group (if present) linking the first reactive group (or precursor thereof) to the acridone ring may be different from the spacer group (if present) linking the second reactive group (or precursor thereof) to the acridone ring.

The spacer group may be a hydrocarbon moiety that may comprise or include one or more moieties selected from aromatic hydrocarbon and aliphatic (straight or branched chain, saturated or unsaturated) moieties, each of which may be interrupted by and/or substituted or terminated by one or more substituents which may optionally include one or more heteroatoms.

Suitable spacer groups include (but are not limited to) one or more of arenediyl, heteroarenediyl, alkanediyl, cycloalkanediyl, heterocycloalkanediyl, aralkanediyl, alkenediyl, alkynediyl, sulphate, sulphonate, mono- or di-substituted amino, —(R_{A})—O—(R_{B})—, —(R_{A})—S—(R_{B})—, —R_{C}—R_{D}—, —(R_{A})—CO₂—(R_{B})—, —(R_{A})—O₂C—(R_{B}), —(R_{A})—CONH—(R_{B})—, —(R_{A})—NHC(O)—(R_{B})—, —(R_{A})—C(O)—(R_{B})—, —(R_{A})—OSO₂O—(R_{B})—, —(R_{A})—SO₂O—(R_{B})—, —(R_{A})—OSO₂—(R_{B})—, —(R_{A})—NH(R_{B})—, —(R_{A})—N(R_{E})(R_{B})—, where R_{A} and R_{B} are optional, and, where present, R_{A}, R_{B}, R_{C} and R_{D} are individually chosen from alkanediyl, arenediyl, alkenediyl, alkynediyl, heteroarenediyl, cycloalkanediyl and heterocycloalkanediyl, and R_{E} is chosen from aryl, heteroaryl, alkyl, cycloalkyl, heterocycloalkyl, aralkyl, alkenyl and alkynyl.

A preferred spacer group between the acridone ring and the first reactive group (or precursor thereof) is an alkanediyl group having between 1 and 12 carbon atoms, preferably —CₙH₂ₙ—, wherein n=1 to 12. This is an especially preferred spacer group if the first reactive group is carboxyl.

It is preferred that the second reactive group (or precursor thereof) is attached to the “2” or “4” position of the acridone (preferably to the “2” position), especially if the second reactive group comprises an amino group (the term “attached” includes the situation where there is a spacer between the reactive group (or precursor thereof) and the acridone ring). For purposes of clarity, the substituent positions used herein are given below in Formula (I):

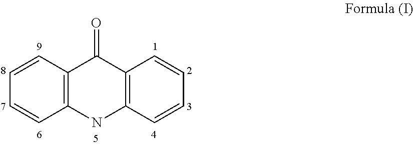

The acridone provided in step (a) preferably comprises a precursor of an amino (—NH₂) group. Such a precursor is preferably attached to the “2” position as shown in Formula I above, and may comprise an substituted amino group, where at least one substituent comprises a protecting group, such as those described above. Once the protecting group is removed, the amino group is the second reactive group.

It is preferred that the first reactive group (or precursor thereof) is attached (optionally via a spacer group) to the “5” position of the acridone ring, especially if the first reactive group comprises an acid group. The spacer group may typically comprise an alkanediyl group (such as CₙH₂ₙ, where n=1 to 12). The acid group preferably comprises carboxyl.

The acridone derivative provided in step (a) may comprise a precursor of the first reactive group. For example, the acridone derivative added in step (a) may comprise an ester group (effectively, an acid group protected by an alkyl or like group) having a general formula COO—Z, wherein Z may be a protecting group, such as dimethoxybenzyl (Dmb), 2-chlorotrityl (Clt) and 2-phenylisopropyl (Pp). Suitable reagents may be used to remove the protecting group to leave a first reactive group, in this case a carboxyl group.

Each of the groups attached to the acridone ring in those positions 1 to 9 not being attached to the first or second reactive groups (or precursors thereof) may be hydrogen, halogen, hydroxyl (—OH), thiol (—SH), cyano (—CN), nitro (—NO₂), —CHO, (but preferably hydrogen) and may include a hydrocarbon moiety that may comprise or include one or more moieties selected from aromatic hydrocarbon and aliphatic (straight or branched chain, saturated or unsaturated) moieties, each of which may be interrupted by and/or substituted or terminated by one or more substituents which may optionally include one or more heteroatoms.

If the acridone derivative provided in step (a) comprises or includes a precursor to the first reactive group, then the method may further comprise treating the acridone derivative provided in step (a) such that first reactive group is formed from the precursor.

Likewise, if the acridone derivative provided in step (a) comprises or includes a precursor to the second reactive group, then the method may further comprise treating the acridone derivative provided in step (a) such that second reactive group is formed from the precursor.

The precursor may comprise a protecting moiety, such as those described above.

The acridone derivative provided in step (a) may have the general structure as shown in Formula (II):

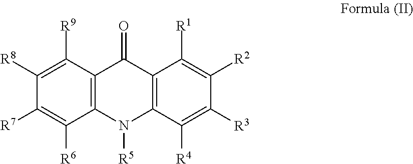

wherein at least two (and preferably only two) of R¹, R², R³, R⁴, R⁵, R⁶, R⁷, R⁸ and R⁹ are independently selected from the list of carboxyl, amino, —(R_{F})—NH(R_{G}), —(R_{F})—NH—SO₂—(R_{G}), —(R_{F})—NH—CO₂—(R_{G}), —(R_{F})—N—(R_{G′}) and the group —X—Y,

wherein X is a linking group and Y is selected from carboxyl, amino, —NH(R_{AT}), —NH—SO₂—(R_{AT}) and —NH—CO₂—(R_{AT}).

wherein R_{F}, R_{G}, R_{G′}, R_{AT} and X may each be a hydrocarbon moiety that may comprise or include one or more moieties selected from aromatic hydrocarbon and aliphatic (straight or branched chain, saturated or unsaturated) moieties, each of which may be interrupted by and/or substituted or terminated by one or more substituents which may optionally include one or more heteroatoms. R_{G}, R_{G′}, R_{AT} may be provided as part of a protecting group as described above with reference to the first aspect of the present invention.

R_{G′} may be a bivalent moiety, such as that which forms a phthalimide group or a diphenylmethyleneamine group.

X is preferably selected from one or more of arenediyl, heteroarenediyl, alkanediyl, cycloalkanediyl, heterocycloalkanediyl, aralkanediyl, alkenediyl, alkynediyl, —(R_{A})—O—(R_{B})—, —(R_{A})—S—(R_{B})—, —R_{C}—R_{D}—, —(R_{A})—CO₂—(R_{B})—, —(R_{A})—O₂C—(R_{B}), —(R_{A})—CONH—(R_{B})—, —(R_{A})—NHC(O)—(R_{B})—, —(R_{A})—C(O)—(R_{C})—, —(R_{A})—OSO₂O—(R_{B})—, —(R_{A})—SO₂O—(R_{B})—, —(R_{A})—OSO₂—(R_{C})—, —(R_{A})—NH(R_{B})—, —(R_{A})—N(R_{E})(R_{B})—, where R_{F}, R_{A} and R_{B} are optional, and, where present, R_{A}, R_{B}, R_{C}, R_{D} and R_{F} are individually chosen from one or more of alkanediyl, arenediyl, alkenediyl, alkynediyl, heteroarenediyl, cycloalkanediyl and heterocycloalkanediyl, R_{E} is chosen from one or more of aryl, heteroaryl, alkyl, cycloalkyl, heterocycloalkyl, aralkyl, alkenyl and alkynyl,

Y is preferably selected from carboxyl, amino, —NH(R_{AT}), —NH—SO₂—(R_{AT}), —NH—CO₂—(R_{AT}) and —N—R_{G′} wherein R_{AT} and R_{G} are individually chosen from one or more of aryl, heteroaryl, alkyl, cycloalkyl, heterocycloalkyl, aralkyl, alkenyl, alkynyl and —R_{UT}—R_{UG} wherein R_{UT} is selected from alkanediyl, arenediyl, alkenediyl, alkynediyl, heteroarenediyl, cycloalkanediyl and heterocycloalkanediyl, R_{UG} is chosen from aryl, heteroaryl, alkyl, cycloalkyl, heterocycloalkyl, aralkyl, alkenyl, alkynyl and R_{G′} is a bivalent moiety capable of binding to nitrogen, such as that which forms a phthalimide group.

The others of R¹, R², R³, R⁴, R⁵, R⁶, R⁷, R⁸ and R⁹ may be independently selected from hydrogen, halogen, hydroxyl (—OH), thiol (—SH), cyano (—CN), nitro (—NO₂), —CHO and a hydrocarbon moiety that may comprise or include one or more moieties selected from aromatic hydrocarbon and aliphatic (straight or branched chain, saturated or unsaturated) moieties, each of which may be interrupted by and/or substituted or terminated by one or more substituents which may optionally include one or more heteroatoms.

The others of R¹, R², R³, R⁴, R⁵, R⁶, R⁷, R⁸ and R⁹ may preferably be independently selected from hydrogen, halogen, hydroxyl (—OH), thiol (—SH), cyano (—CN), nitro (—NO₂), —CHO, and one or more of aryl, heteroaryl, alkyl, cycloalkyl, heterocycloalkyl, aralkyl, alkenyl, alkynyl, —(R_{AA})—O—(R_{BB}), —(R_{AA})—S—(R_{BB}), —R_{CC}—R_{DD}, —(R_{AA})—CO₂—(R_{BB}), —(R_{AA})—O₂C—(R_{BB}), —(R_{AA})—CONH—(R_{BB}), —(R_{AA})—NHC(O)—(R_{BB}), —(R_{AA})—C(O)—(R_{BB}), —(R_{AA})—OSO₂O—(R_{BB}), —(R_{AA})—SO₂O—(R_{BB}), —(R_{AA})—OSO₂—(R_{BB}), —(R_{AA})—NH(R_{BB}), —(R_{AA})—N(R_{BB})(R_{EE}), —(R_{AA})—SO₂OH where R_{AA} is optional, and, where present, R_{AA} and R_{CC} are individually chosen from one or more of alkanediyl, arenediyl, alkenediyl, alkynediyl, heteroarenediyl, cycloalkanediyl and heterocycloalkanediyl, and R_{BB}, R_{DD} and R_{EE} are individually chosen from one or more of aryl, heteroaryl, alkyl, cycloalkyl, heterocycloalkyl, aralkyl, alkenyl and alkynyl.

and the group -E-F,

wherein E is a linking group selected from one or more of arenediyl, heteroarenediyl, alkanediyl, cycloalkanediyl, heterocycloalkanediyl, aralkanediyl, alkenediyl, alkynediyl, —(R_{H})—O—(R_{I})—, —(R_{H})—S—(R_{I})—, —R_{J}—R_{K}—, —(R_{H})—CO₂—(R_{I})—, —(R_{H})—O₂C—(R_{I})—, —(R_{H})—CONH—(R_{I})—, —(R_{H})—NHC(O)—(R_{I})—, —(R_{H})—C(O)—(R_{I})—, —(R_{H})—OSO₂O—(R_{I})—, —(R_{H})—SO₂O—(R_{I})—, —(R_{H})—OSO₂—(R_{I})—, —(R_{H})—NH(R_{I})—, —(R_{H})—N(R_{L})(R_{I})—, where R_{H} and R_{I} are optional, and, where present, R_{H}, R_{I}, R_{J} and R_{K} are individually chosen from one or more of alkanediyl, arenediyl, alkenediyl, alkynediyl, heteroarenediyl, cycloalkanediyl and heterocycloalkanediyl, and R_{L} is chosen from one or more of aryl, heteroaryl, alkyl, cycloalkyl, heterocycloalkyl, aralkyl, alkenyl and alkynyl,

and F is selected from —(R_{M})—OSO₂O—(R_{N}), —(R_{M})—SO₂O—(R_{N.}), —(R_{M})—OSO₂—(R_{N}), —(R_{M})—CO₂—(R_{N}), —(R_{M})—O₂C—(R_{N}), —(R_{M})—N(R_{N})(R_{O}), where R_{M} is optional, and where present is chosen from one or more of alkanediyl, arenediyl, alkenediyl, alkynediyl, heteroarenediyl, cycloalkanediyl and heterocycloalkanediyl, and where R_{N} and R_{O} are individually chosen from one or more of aryl, heteroaryl, alkyl, cycloalkyl, heterocycloalkyl, aralkyl, alkenyl and alkynyl.

The at least two of R¹, R², R³, R⁴, R⁵, R⁶, R⁷, R⁸ and R⁹ provide two reactive groups (first and second reactive groups), one for reaction with the solid support and one for reaction with constituent peptides so as to form a fluorogenic peptide.

It is preferred that R⁵ is —(CₙH₂ₙ—CO₂H), R²═—NH(R_{AT}), wherein R_{AT} is a protecting moiety (such as Fmoc), the rest of R¹ to R⁹ being H.

For the avoidance of confusion, the terms used in the specification are described.

“Carboxyl” is —CO₂H.

“Halogen” refers to fluoro, chloro, bromo and iodo groups.

“Aryl” refers to a monovalent unsaturated aromatic carbocyclic groups having single or multiple rings, preferably having from 5 to 20 carbon atoms. Examples of aryl groups include phenyl, biphenyl and naphthyl. The aryl group may optionally be substituted with one or more halogen or hydroxyl (—OH) groups.

“Heteroaryl” refers to a monovalent heteroaromatic group. The group may, for example, be monocyclic, bicyclic or tricyclic. The rings may be fused. Examples of heteroaryl groups include pyridyl, thienyl and furyl. The heteroaryl group may optionally be substituted with one or more halogen or hydroxyl (—OH) groups.

“Alkyl” includes straight and branched alkyl groups, preferably having up to 12 carbon atoms and more preferably having from 1 to 6 carbon atoms. This term is exemplified by groups such as methyl (—CH₃), ethyl (—C₂H₅), n-propyl, isopropyl, n-butyl, isobutyl and tert-butyl (—C(CH₃)₃). The alkyl group may optionally be substituted with one or more halogen or hydroxyl (—OH) groups.

“Alkenyl” refers to monovalent groups having at least 1 site of alkenyl unsaturation, and preferably having up to 10 carbon atoms, more preferably from 2 to 6 carbon atoms. Such groups may, for example, vinyl (—CH═CH₂), propenyl (—CH═CH(CH₃)) or butenyl. The alkenyl group may optionally be substituted with one or more halogen or hydroxyl (—OH) groups.

“Alkynyl” refers to monovalent groups having at least 1 site of alkynyl unsaturation, and preferably having up to 10 carbon atoms, more preferably from 2 to 6 carbon atoms. Such groups, may, for example, be propynyl (—CH₂CCH) or butynyl.

The alkynyl group may optionally be substituted with one or more halogen or hydroxyl (—OH) groups.

“Cycloalkyl” refers to a monovalent saturated carbocyclic group having one or more rings. The group may preferably comprise from 3 to 10 carbon atoms. Examples of such groups include cyclohexyl (—C₆H₁₁) and cyclopentyl (—C₅H₉). The cycloalkyl group may optionally be substituted with one or more halogen or hydroxyl (—OH) groups.

“Heterocycloalkyl” refers to a monovalent saturated cyclic group comprising one or more heteroatom. An example of such a group is piperidinyl. The heterocycloalkyl group may optionally be substituted with one or more halogen or hydroxyl (—OH) groups.

“Aralkyl” refers to a monovalent group comprising an aryl group bonded through an alkanediyl group. Examples of such groups are benzyl (—CH₂—C₆H₅) or phenethyl. The aralkyl group may optionally be substituted with one or more halogen or hydroxyl (—OH) groups.

“Amino” is —NH₂.

“Carbonyl” is a divalent —CO— moiety or group.

“Alkanediyl” is a divalent group, exemplified by groups such as methandiyl (—CH₂—), ethandiyl (—CH₂—CH₂—), propan-1,3-diyl, propan-1,2-diyl and butan-1,4-diyl. The term includes straight and branched alkanediyl groups, preferably having up to 12 carbon atoms and more preferably having from 1 to 6 carbon atoms. The alkanediyl group may optionally be substituted with one or more halogen or hydroxyl (—OH) groups.

“Cycloalkanediyl” refers to a divalent saturated carbocyclic group having one or more rings. The group may preferably comprise from 3 to 10 carbon atoms. Examples of such groups include cyclohexan-1,4-diyl and cyclopentan-1,3-diyl. The cycloalkanediyl group may optionally be substituted with one or more halogen or hydroxyl (—OH) groups.

“Alkenediyl” refers to divalent groups having at least 1 site of alkenyl unsaturation, and preferably having up to 10 carbon atoms, more preferably from 2 to 6 carbon atoms. An example of such groups is ethen-1,2-diyl (—CH═CH—). The alkenediyl group may optionally be substituted with one or more halogen or hydroxyl (—OH) groups.

“Alkynediyl” refers to divalent groups having at least 1 site of alkynyl unsaturation, and preferably having up to 10 carbon atoms, more preferably from 2 to 6 carbon atoms. An examples of such groups is ethyn-1,2-diyl (—C≡C—). The alkynediyl group may optionally be substituted with one or more halogen or hydroxyl (—OH) groups.

“Arenediyl” refers to divalent unsaturated aromatic carbocyclic groups having single or multiple rings, preferably having from 5 to 20 carbon atoms. Examples of aryl groups include phenylene, biphenyl-diyl and naphthalene-4a,8a-diyl. The arenediyl group may optionally be substituted with one or more halogen or hydroxyl (—OH) groups.

“Heteroarenediyl” refers to a divalent heteroaromatic group. The group may, for example, be monocyclic, bicyclic or tricyclic. The rings may be fused. Examples of such groups include pyridylene, thienylene and furylene. The heteroarenediyl group may optionally be substituted with one or more halogen or hydroxyl (—OH) groups.

“Heterocycloalkanediyl” refers to a divalent saturated cyclic group comprising one or more heteroatom, such as 1,4 dioxane-2,3-diyl. The heterocycloalkanediyl group may optionally be substituted with one or more halogen or hydroxyl (—OH) groups.

It is preferred that at least one of R¹, R², R³, R⁴, R⁵, R⁶, R⁷, R⁸ and R⁹ (and preferably R⁴, R⁵ or R⁶, more preferably R⁵) is selected from the list of carboxyl, and the group —X—Y,

wherein X is a linking group that includes a hydrocarbon moiety that may comprise or include one or more moieties selected from aromatic hydrocarbon and aliphatic (straight or branched chain, saturated or unsaturated) moieties, each of which may be interrupted by and/or substituted or terminated by one or more substituents which may optionally include one or more heteroatoms,

and Y is carboxyl.

More preferably, at least one of R¹, R², R³, R⁴, R⁵, R⁶, R⁷, R⁸ and R⁹ (and preferably R⁴, R⁵ or R⁶, more preferably R⁵) is selected from the list of carboxyl, and the group —X—Y, wherein X is selected from one or more of arenediyl, heteroarenediyl, alkanediyl, cycloalkanediyl, heterocycloalkanediyl, aralkanediyl, alkenediyl, alkynediyl, —(R_{A})—O—(R_{B})—, —(R_{A})—S—(R_{B})—, —R_{C}—R_{D}—, —(R_{A})—CO₂—(R_{B})—, —(R_{A})—CONH—(R_{B})—, —(R_{A})—C(O)—(R_{C})—, —(R_{A})—OSO₂O—(R_{B})—, —(R_{A})—SO₂O—(R_{B})—, —(R_{A})—NH(R_{B})—, —(R_{A})—N(R_{E})(R_{B})—, where R_{F}, R_{A} and R_{B} are optional, and, where present, R_{A}, R_{B}, R_{C}, R_{D} and R_{F} are individually chosen from one or more of alkanediyl, arenediyl, alkenediyl, alkynediyl, heteroarenediyl, cycloalkanediyl and heterocycloalkanediyl, and R_{E} is individually chosen from one or more of aryl, heteroaryl, alkyl, cycloalkyl, heterocycloalkyl, aralkyl, alkenyl and alkynyl,

and Y is carboxyl.

This provides an effective first reactive group for reaction with the solid.

The linking group, X, may preferably be selected from one or more of arenediyl, heteroarenediyl, alkanediyl, cycloalkanediyl, aralkanediyl, alkenediyl, alkynediyl and —R_{C}—R_{D}—, where R_{C} and R_{D} are individually chosen from one or more of alkanediyl, arenediyl, alkenediyl, alkynediyl, heteroarenediyl, cycloalkanediyl and heterocycloalkanediyl.

The linking group is preferably selected from alkanediyl and alkenediyl, such linking group preferably having from 2 to 10 carbon atoms. It is preferred that X is alkenediyl, preferably having from 2 to 10 carbon atoms, further preferably having from 3 to 8 carbon atoms.

It is preferred that at least one of R¹, R², R³, R⁴, R⁵, R⁶, R⁷, R⁸ and R⁹ (and preferably R², R⁴, R⁶ or R⁸, more preferably R²) is selected from the list of amino, —(R_{F})—NH(R_{G}), —(R_{F})—NH—SO₂—(R_{G}), —(R_{F})—NH—CO₂—(R_{G}), —(R_{F})—N—R_{G′} and —X—Y,

wherein X is a linking group and Y is selected from carboxyl, amino and —NH(R_{AT}), —NH—SO₂—(R_{AT}), —NH—CO₂—(R_{AT})

wherein X, R_{AT}, R_{G} and R_{G′} include a hydrocarbon moiety that may comprise or include one or more moieties selected from aromatic hydrocarbon and aliphatic (straight or branched chain, saturated or unsaturated) moieties, each of which may be interrupted by and/or substituted or terminated by one or more substituents which may optionally include one or more heteroatoms,

Y may comprise a phthalimide group.

X is preferably selected from one or more of arenediyl, heteroarenediyl, alkanediyl, cycloalkanediyl, heterocycloalkanediyl, aralkanediyl, alkenediyl, alkynediyl, —(R_{A})—O—(R_{B})—, —(R_{A})—S—(R_{B})—, —R_{C}—R_{D}—, —(R_{A})—CO₂—(R_{B})—, —(R_{A})—CONH—(R_{B})—, —(R_{A})—C(O)—(R_{C})—, —(R_{A})—OSO₂O—(R_{B})—, —(R_{A})—SO₂O—(R_{B})—, —(R_{A})—NH(R_{B})—, —(R_{A})—N(R_{E})(R_{B})—, where R_{F}, R_{A} and R_{B} are optional, and, where present, R_{A}, R_{B}, R_{C}, R_{D} and R_{F} are individually chosen from one or more of alkanediyl, arenediyl, alkenediyl, alkynediyl, heteroarenediyl, cycloalkanediyl and heterocycloalkanediyl, and R_{E} is chosen from aryl, heteroaryl, alkyl, cycloalkyl, heterocycloalkyl, aralkyl, alkenyl and alkynyl,

and Y may be selected from carboxyl, amino and —NH(R_{AT}), —NH—SO₂—(R_{AT}), —NH—CO₂—(R_{AT}), —N—R_{G′} wherein R_{AT} and R_{G} are individually chosen from aryl, heteroaryl, alkyl, cycloalkyl, heterocycloalkyl, aralkyl, alkenyl, alkynyl and —R_{UT}—R_{UG} wherein R_{UT} is selected from one or more of alkanediyl, arenediyl, alkenediyl, alkynediyl, heteroarenediyl, cycloalkanediyl and heterocycloalkanediyl, and R_{UG} is chosen from aryl, heteroaryl, alkyl, cycloalkyl, heterocycloalkyl, aralkyl, alkenyl, alkynyl, and R_{G′} is a bivalent group capable of binding to nitrogen. R_{G′} may be, for example, the group that forms a phthalimide group.

It is preferred that the said at least one of R¹, R², R³, R⁴, R⁵, R⁶, R⁷, R⁸ and R⁹ (and preferably R², R⁴, R⁶ or R⁸, more preferably R²) comprises —(R_{F})—NH(R_{G}), —(R_{F})—NH—SO₂—(R_{G}), —(R_{F})—NH—CO₂—(R_{G}) or —(R_{F})—N—R_{G′} as defined above.

It is preferred that the said at least one of R¹, R², R³, R⁴, R⁵, R⁶, R⁷, R⁸ and R⁹ (and preferably R², R⁴, R⁶ or R⁸, more preferably R²) comprises —(R_{F})—NH—SO₂—(R_{G}) or —(R_{F})—NH—CO₂—(R_{G}). It is preferred, in this case, that R_{F} is absent and R_{G} is alkyl or aralkyl.

If at least one of R¹, R², R³, R⁴, R⁵, R⁶, R⁷, R⁸ and R⁹ (and preferably R², R⁴, R⁶ or R⁸, more preferably R²) is —X—Y, where X is a linking group as defined above and Y is amino, —NH(R_{AT}), —NH—SO₂—(R_{AT}), —NH—CO₂—(R_{AT}) or —N—R_{G′} as defined above, then it is preferred that X is alkanediyl. The alkanediyl may preferably comprise between 2 and 10 carbon atoms.

This provides an effective second reactive group for the formation of a fluorogenic peptide.

It is preferred that the others of R¹, R², R³, R⁴, R⁵, R⁶, R⁷, R⁸ and R⁹ not forming the first and second reactive groups are independently selected from hydrogen, halogen, hydroxyl (—OH), thiol (—SH), cyano (—CN), nitro (—NO₂), —CHO, aryl, heteroaryl, alkyl, cycloalkyl, heterocycloalkyl, aralkyl, alkenyl, alkynyl, —(R_{AA})—O—(R_{BB}), —(R_{AA})—S—(R_{BB}), —R_{CC}—R_{DD}, —(R_{AA})—CO₂—(R_{BB}), —(R_{AA})—O₂C—(R_{BB}), —(R_{AA})—CONH—(R_{BB}), —(R_{AA})—NHC(O)—(R_{BB}), —(R_{AA})—C(O)—(R_{BB}), —(R_{AA})—OSO₂O—(R_{BB}), —(R_{AA})—SO₂O—(R_{BB}), —(R_{AA})—OSO₂—(R_{BB}), —(R_{AA})—NH(R_{BB}), —(R_{AA})—N(R_{BB})(R_{EE}), —(R_{AA})—SO₂OH where R_{AA} is optional, and, where present, R_{AA} and R_{CC} are individually chosen from one or more of alkanediyl, arenediyl, alkenediyl, alkynediyl, heteroarenediyl, cycloalkanediyl and heterocycloalkanediyl, and R_{BB}, R_{DD} and R_{EE} are individually chosen from aryl, heteroaryl, alkyl, cycloalkyl, heterocycloalkyl, aralkyl, alkenyl and alkynyl.

More preferably, the others of R¹, R², R³, R⁴, R⁵, R⁶, R⁷, R⁸ and R⁹ not forming the first and second reactive groups are independently selected from hydrogen, halogen, hydroxyl (—OH), thiol (—SH), cyano (—CN), nitro (—NO₂) and alkyl.

Most preferably, the others of R¹, R², R³, R⁴, R⁵, R⁶, R⁷, R⁸ and R⁹ not forming the first and second reactive groups are hydrogen or alkyl comprising 1 to 10 carbon atoms, more preferably hydrogen.

Step (c) may be achieved by reacting the first reactive group of the acridone derivative, typically an acid group, with a suitably prepared solid support. Such solid supports are well-known to those skilled in the art of solid phase peptide synthesis. A coupling reagent may be used to attach the acridone derivative to the solid support.

The acid may be attached to a solid phase resin, for example a 2-chlorotrityl chloride resin. This reaction does not require the use of activating reagents and is carried out in the presence of a base such as diisopropyldiethylamine (DIEA). Any unreacted chloro groups remaining on the resin after coupling are capped by reacting with methanol/DIEA.

An alternative method is to use hydroxyl containing resins. The acid may be attached to a solid phase resin, for example a hydroxyl containing resin such as those derivatised with p-benzyl alcohol (e.g. Wang and NovaPEG resins) via an ester linkage. This attachment can be effected by using a coupling agent such as 1-(mesitylene-2-sulphonyl)-3-nitro-1H-1,2,4-triazole (MSNT) in the presence of a catalyst such as N,N-dimethylaminopyridine (DMAP) as described by Blankemeyer-Menge et al., “An Efficient Method For Anchoring Fmoc-Amino Acids To Hydroxyl-Functionalised Solid Supports”, Tetrahedron Letters, vol. 21, 12, (1990) pp. 1701-1704.

Once the acridone derivative has been attached to the solid support, peptide synthesis may take place. The reagents and conditions for such synthesis are well-known to those skilled in the art of solid phase peptide synthesis. For example, stepwise elongation of the peptide may be performed by sequential addition of Fmoc protected amino acids using condensing agents such as benzotriazole-1-yl-oxy-tris-pyrrolidino-phosphonium hexafluorophosphate (PyBOP®) and 2-(1-H-9-azobenzotriazole-1-yl)-1,1,3,3-tetramethylaminium hexafluorophosphate (HATU) in the presence of a base such as diisopropylethylamine (DIEA).

Upon completion of the synthesis the peptide may be cleaved from the solid support. The reaction conditions used to effect this may also cleave side chain protecting groups from one or more amino acid moieties. The fully deprotected peptide may then be purified, for exampled by using HPLC.

Alternatively or additionally, fragment condensation may be used, especially if it is desired to produce longer peptides.

This may be carried out by synthesising fragments of a long peptide chain on an acid-labile resin, such as a chlorotrityl resin. The fragments can be cleaved from the resin under mild acidic conditions that leave the side chain protecting groups of the amino acids intact. The fragments may then be condensed sequentially to give the full length peptide which is then fully deprotected. Examples are described by V. \check{C}e\check{r}ovský and H. A. Scheraga, Journal of Peptide Research, vol. 65, (2005) pp. 518-528.

If the acridone derivative provided in step (a) comprises a protective moiety, for example an Fmoc group protecting an amino group, then step (e) may comprise removing the protecting group.

The peptide may typically comprise from 1 to 20 amino acid residues, preferably 1 to 10 residues and more preferably 4. to 10 residues. It is preferred that the peptide comprises at least one residue of one or more of tyrosine, nitrotyrosine, nitrophenylalanine and tryptophan. It is preferred that the fluorophor is attached to one terminus of the peptide.

In accordance with a second aspect of the present invention there is provided a compound of formula (II),

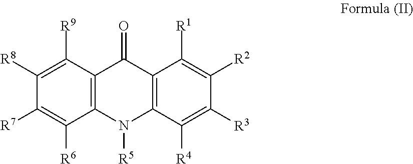

at least one of R⁴, R⁵ and R⁶ (preferably R⁵) is independently selected from the list of carboxyl, amino, —(R_{F})—NH(R_{G}), —(R_{F})—NH—SO₂—(R_{G}), —(R_{F})—NH—CO₂—(R_{G}), —(R_{F})—N—R_{G′} and the group —X—Y,

wherein X is a linking group selected from arenediyl, heteroarenediyl, alkanediyl, cycloalkanediyl, heterocycloalkanediyl, aralkanediyl, alkenediyl, alkynediyl, heterocycloalkyl, aralkyl, alkenyl, alkynyl, —(R_{AA})—O—(R_{BB}), —(R_{A})—O—(R_{B})—, —(R_{A})—S—(R_{B})—, —R_{C}—R_{D}—, —(R_{A})—CO₂—(R_{B})—, —(R_{A})—O₂C—(R_{B}), —(R_{A})—CONH—(R_{B})—, —(R_{A})—NHC(O)—(R_{B})—, —(R_{A})—C(O)—(R_{C})—, —(R_{A})—OSO₂O—(R_{B})—, —(R_{A})—SO₂O—(R_{B})—, —(R_{A})—OSO₂—(R_{C})—, —(R_{A})—NH(R_{B})—, —(R_{A})—N(R_{E})(R_{B})—, where R_{F}, R_{A} and R_{B} are optional, and, where present, R_{A}, R_{B}, R_{C}, R_{D} and R_{F} are individually chosen from alkanediyl, arenediyl, alkenediyl, alkynediyl, heteroarenediyl, cycloalkanediyl and heterocycloalkanediyl, and R_{E} is chosen from aryl, heteroaryl, alkyl, cycloalkyl, heterocycloalkyl, aralkyl, alkenyl and alkynyl,

and Y is selected from carboxyl, amino, —NH(R_{AT}), —NH—SO₂—(R_{AT}), —NH—CO₂—(R_{AT}) and —N—R_{G′} wherein R_{AT} and R_{G} are each hydrocarbon moieties that may comprise or include one or more moieties selected from aromatic hydrocarbons and aliphatic (straight or branched chain, saturated or unsaturated) moieties, each of which may be interrupted by and/or substituted by one or more substituents which may optionally include one or more heteroatoms, and R_{G′} is a bivalent group capable of binding to nitrogen.

R_{G′}, R_{AT} and R_{G} may be provided as a protecting group.

R_{AT} and R_{G} may be individually chosen from aryl, heteroaryl, alkyl, cycloalkyl, heterocycloalkyl, aralkyl, alkenyl and alkynyl and —R_{UT}—R_{UG}, wherein R_{UT} is selected from alkanediyl, arenediyl, alkenediyl, alkynediyl, heteroarenediyl, cycloalkanediyl and heterocycloalkanediyl, R_{UG} is chosen from aryl, heteroaryl, alkyl, cycloalkyl, heterocycloalkyl, aralkyl, alkenyl, alkynyl.

R_{G′} may typically be the group that, when bound to the nitrogen atom, forms a phthalimide group.

And at least one of R², R⁴, R⁶ and R⁸ (preferably R²) is selected from the list of amino, —(R_{F})—NH(R_{G}), —(R_{F})—NH—SO₂—(R_{G}), —(R_{F})—NH—CO₂—(R_{G}), —(R_{F})—N—R_{G′} and the group —X—Y,

Wherein X is a linking group selected from arenediyl, heteroarenediyl, alkanediyl, cycloalkanediyl, heterocycloalkanediyl, aralkanediyl, alkenediyl, alkynediyl, —(R_{A})—O—(R_{B})—, —(R_{A})—S—(R_{B})—, —R_{C}—R_{D}—, —(R_{A})—CO₂—(R_{B})—, —(R_{A})—O₂C—(R_{B}), —(R_{A})—CONH—(R_{B})—, —(R_{A})—NHC(O)—(R_{B})—, —(R_{A})—C(O)—(R_{C})—, —(R_{A})—OSO₂O—(R_{B})—, —(R_{A})—SO₂O—(R_{B})—, —(R_{A})—OSO₂—(R_{C})—, —(R_{A})—NH(R_{B})—, —(R_{A})—N(R_{E})(R_{B})—, where R_{F}, R_{A} and R_{B} are optional, and, where present, R_{A}, R_{B}, R_{C}, R_{D} and R_{F} are individually chosen from alkanediyl, arenediyl, alkenediyl, alkynediyl, heteroarenediyl, cycloalkanediyl and heterocycloalkanediyl, and R_{E} is chosen from aryl, heteroaryl, alkyl, cycloalkyl, heterocycloalkyl, aralkyl, alkenyl and alkynyl and

Y is selected from amino, —NH(R_{AT}) , —NH—SO₂—(R_{AT}), —NH—CO₂—(R_{AT}) and —N—R_{G′}, wherein R_{AT} and R_{G} are individually chosen from aryl, heteroaryl, alkyl, cycloalkyl, heterocycloalkyl, aralkyl, alkenyl and alkynyl, and —R_{UT}—R_{UG} wherein R_{UT} is selected from alkanediyl, arenediyl, alkenediyl, alkynediyl, heteroarenediyl, cycloalkanediyl and heterocycloalkanediyl, R_{UG} is chosen from aryl, heteroaryl, alkyl, cycloalkyl, heterocycloalkyl, aralkyl, alkenyl, alkynyl and R_{G′} is a bivalent group capable of binding to nitrogen

where the others of R¹, R², R³, R⁴, R⁵, R⁶, R⁷, R⁸ and R⁹ are independently selected from hydrogen, halogen, hydroxyl (—OH), thiol (—SH), cyano (—CN), nitro (—NO₂), —CHO, aryl, heteroaryl, alkyl, cycloalkyl, heterocycloalkyl, aralkyl, alkenyl, alkynyl, —(R_{AA})—O—(R_{BB}), —(R_{AA})—S—(R_{BB}), —R_{CC}—R_{DD}, —(R_{AA})—CO₂—(R_{BB}), —(R_{AA})—O₂C—(R_{BB}), —(R_{AA})—CONH—(R_{BB}), —(R_{AA})—NHC(O)—(R_{BB}), —(R_{AA})—C(O)—(R_{BB}), —(R_{AA})—OSO₂O—(R_{BB}), —(R_{AA})—SO₂O—(R_{BB}), —(R_{AA})—OSO₂—(R_{BB}), —(R_{AA})—NH(R_{BB}), —(R_{AA})—N(R_{BB})(R_{EE}), —(R_{AA})—SO₂OH where R_{AA} is optional, and, where present, R_{AA} and R_{CC} are individually chosen from alkanediyl, arenediyl, alkenediyl, alkynediyl, heteroarenediyl, cycloalkanediyl and heterocycloalkanediyl, and R_{BB}, R_{DD} and R_{EE} are individually chosen from aryl, heteroaryl, alkyl, cycloalkyl, heterocycloalkyl, aralkyl, alkenyl and alkynyl.

and the group -E-F,

wherein E is a linking group selected from arenediyl, heteroarenediyl, alkanediyl, cycloalkanediyl, heterocycloalkanediyl, aralkanediyl, alkenediyl, alkynediyl, —(R_{H})—O—(R_{I})—, —(R_{H})—S—(R_{I})—, —R_{J}—R_{K}—, —(R_{H})—CO₂—(R_{I})—, —(R_{H})—O₂C—(R_{I})—, —(R_{H})—CONH—(R_{I})—, —(R_{H})—NHC(O)—(R_{I})—, —(R_{H})—C(O)—(R_{I})—, —(R_{H})—OSO₂O—(R_{I})—, —(R_{H})—SO₂O—(R_{I})—, —(R_{H})—OSO₂—(R_{I})—, —(R_{H})—NH(R_{I})—, —(R_{H})—N(R_{L})(R_{I})—, where R_{H} and R_{I} are optional, and, where present, R_{H}, R_{I}, R_{J} and R_{K} are individually chosen from alkanediyl, arenediyl, alkenediyl, alkynediyl, heteroarenediyl, cycloalkanediyl and heterocycloalkanediyl, and R_{L} is chosen from aryl, heteroaryl, alkyl, cycloalkyl, heterocycloalkyl, aralkyl, alkenyl and alkynyl,

and F is selected from —(R_{M})—OSO₂O—(R_{N}), —(R_{M})—SO₂O—(R_{N}), —(R_{M})—OSO₂—(R_{N}), —(R_{M})—CO₂—(R_{N}), —(R_{M})—O₂C—(R_{N}), —(R_{M})—N(R_{N})(R_{O}), where R_{M} is optional, and where present is chosen from alkanediyl, arenediyl, alkenediyl, alkynediyl, heteroarenediyl, cycloalkanediyl and heterocycloalkanediyl, and where R_{N} and R_{O} are individually chosen from aryl, heteroaryl, alkyl, cycloalkyl, heterocycloalkyl, aralkyl, alkenyl and alkynyl.

The at least one of R⁴, R⁵ and R⁶ forms a first reactive group for forming a link between the acridone derivative and the solid phase support.

The at least one of R², R⁴, R⁶ and R⁸ forms a second reactive group for forming a template for the formation of a peptide.

It is preferred that at least one of R¹, R², R³, R⁴, R⁵, R⁶, R⁷, R⁸ and R⁹ (and preferably R², R⁴, R⁶ or R⁸, more preferably R²) is selected from the list of amino, —(R_{F})—NH(R_{G}), —(R_{F})—NH—SO₂—(R_{G}), —(R_{F})—NH—CO₂—(R_{G}), —(R_{F})—N—R_{G′} and —X—Y,

wherein X is a linking group selected from arenediyl, heteroarenediyl, alkanediyl, cycloalkanediyl, heterocycloalkanediyl, aralkanediyl, alkenediyl, alkynediyl, —(R_{A})—O—(R_{B})—, —(R_{A})—S—(R_{B})—, —R_{C}—R_{D}—, —(R_{A})—CO₂—(R_{B})—, —(R_{A})—O₂C—(R_{B}), —(R_{A})—CONH—(R_{B})—, —(R_{A})—NHC(O)—(R_{B})—, —(R_{A})—C(O)—(R_{C})—, —(R_{A})—OSO₂O—(R_{B})—, —(R_{A})—SO₂O—(R_{B})—, —(R_{A})—OSO₂—(R_{C})—, —(R_{A})—NH(R_{B})—, —(R_{A})—N(R_{E})(R_{B})—, where R_{F}, R_{A} and R_{B} are optional, and, where present, R_{A}, R_{B}, R_{C}, R_{D} and R_{F} are individually chosen from alkanediyl, arenediyl, alkenediyl, alkynediyl, heteroarenediyl, cycloalkanediyl and heterocycloalkanediyl, R_{E} is chosen from aryl, heteroaryl, alkyl, cycloalkyl, heterocycloalkyl, aralkyl, alkenyl and alkynyl, and R_{G′} is a bivalent group capable of binding to nitrogen

and Y is selected from carboxyl, amino and —NH(R_{AT}), —NH—SO₂—(R_{AT}), —NH—CO₂—(R_{AT}), —N—R_{G′} wherein R_{AT} and R_{G} are individually chosen from aryl, heteroaryl, alkyl, cycloalkyl, heterocycloalkyl, aralkyl, alkenyl, alkynyl and —R_{UT}—R_{UG} wherein R_{UT} is selected from alkanediyl, arenediyl, alkenediyl, alkynediyl, heteroarenediyl, cycloalkanediyl and heterocycloalkanediyl, R_{UG} is chosen from aryl, heteroaryl, alkyl, cycloalkyl, heterocycloalkyl, aralkyl, alkenyl, alkynyl, and R_{G′} is a bivalent group capable of binding to nitrogen;.

It is preferred that the said at least one of R¹, R², R³, R⁴, R⁵, R⁶, R⁷, R⁸ and R⁹ (and preferably R², R⁴, R⁶ or R⁸, more preferably R²) comprises —(R_{F})—NH(R_{G}), —(R_{F})—NH—SO₂—(R_{G}), —(R_{F})—NH—CO₂—(R_{G}) or —(R_{F})—N—R_{G′} as defined above.

It is preferred that the said at least one of R¹, R², R³, R⁴, R⁵, R⁶, R⁷, R⁸ and R⁹ (and preferably R², R⁴, R⁶ or R⁸, more preferably R²) comprises —(R_{F})—NH—SO₂—(R_{G}) or —(R_{F})—NH—CO₂—(R_{G}). It is preferred, in this case, that R_{F} is absent and R_{G} is alkyl or aralkyl.

It is preferred that the at least one of R⁴, R⁵ and R⁶ is selected from the list of carboxyl, and the group —X—Y, wherein X is a linking group selected from arenediyl, heteroarenediyl, alkanediyl, cycloalkanediyl, heterocycloalkanediyl, aralkanediyl, alkenediyl, alkynediyl, —(R_{A})—O—(R_{B})—, —(R_{A})—S—(R_{B})—, —R_{C}—R_{D}—, —(R_{A})—CO₂—(R_{B})—, —(R_{A})—O₂C—(R_{B}), —(R_{A})—CONH—(R_{B})—, —(R_{A})—NHC(O)—(R_{B})—, —(R_{A})—C(O)—(R_{C})—, —(R_{A})—OSO₂O—(R_{B})—, —(R_{A})—SO₂O—(R_{B})—, —(R_{A})—OSO₂—(R_{C})—, —(R_{A})—NH(R_{B})—, —(R_{A})—N(R_{E})(R_{B})—, where R_{F}, R_{A} and R_{B} are optional, and, where present, R_{A}, R_{B}, R_{C}, R_{D} and R_{F} are individually chosen from alkanediyl, arenediyl, alkenediyl, alkynediyl, heteroarenediyl, cycloalkanediyl and heterocycloalkanediyl, and R_{E} and R_{G} are individually chosen from aryl, heteroaryl, alkyl, cycloalkyl, heterocycloalkyl, aralkyl, alkenyl and alkynyl

and Y is carboxyl. X is preferably alkanediyl, more preferably comprising between 2 and 10 carbon atoms.

It is preferred that the others of R¹, R², R⁴, R⁵, R⁶, R⁸ and R⁹ not forming the first and second reactive groups are independently selected from hydrogen, halogen, hydroxyl (—OH), thiol (—SH), cyano (—CN), nitro (—NO₂), —CHO, aryl, heteroaryl, alkyl, cycloalkyl, heterocycloalkyl, aralkyl, alkenyl, alkynyl, —(R_{AA})—O—(R_{BB}), —(R_{AA})—S—(R_{BB}), —R_{CC}—R_{DD}, —(R_{AA})—CO₂—(R_{BB}), —(R_{AA})—O₂C—(R_{BB}), —(R_{AA})—CONH—(R_{BB}), —(R_{AA})—NHC(O)—(R_{BB}), —(R_{AA})—C(O)—(R_{BB}), —(R_{AA})—OSO₂O—(R_{BB}), —(R_{AA})—SO₂O—(R_{BB}), —(R_{AA})—OSO₂—(R_{BB}), —(R_{AA})—NH(R_{BB}), —(R_{AA})—N(R_{BB})(R_{EE}), —(R_{AA})—SO₂OH where R_{AA} is optional, and, where present, R_{AA} and R_{CC} are individually chosen from one or more of alkenediyl, alkynediyl, heteroarenediyl, cycloalkanediyl and heterocycloalkanediyl, and R_{BB}, R_{DD} and R_{EE} are individually chosen from aryl, heteroaryl, alkyl, cycloalkyl, heterocycloalkyl, aralkyl, alkenyl and alkynyl.

More preferably, the others of R¹, R², R⁴, R⁵, R⁶, R⁸ and R⁹ not forming the first and second reactive groups are independently selected from hydrogen, halogen, hydroxyl (—OH), thiol (—SH), cyano (—CN), nitro (—NO₂) and alkyl.

Most preferably, the others of R¹, R², R⁴, R⁵, R⁶, R⁸ and R⁹ not forming the first and second reactive groups are hydrogen.

In accordance with a third aspect of the present invention there is provided a solid support to which a fluorogenic reporter is attached, the fluorogenic reporter being an acridone derivative.

It is preferred that the acridone is linked to the solid support via a linking group, the linking group comprising one or more of arenediyl, heteroarenediyl, alkanediyl, cycloalkanediyl, heterocycloalkanediyl, aralkanediyl, alkenediyl, alkynediyl, —(R_{A})—O—(R_{B})—, —(R_{A})—S—(R_{B})—, —R_{C}—R_{D}—, —(R_{A})—CO₂—(R_{B})—, —(R_{A})—O₂C—(R_{B}), —(R_{A})—CONH—(R_{B})—, —(R_{A})—NHC(O)—(R_{B})—, —(R_{A})—C(O)—(R_{C})—, —(R_{A})—OSO₂O—(R_{B})—, —(R_{A})—SO₂O—(R_{B})—, —(R_{A})—OSO₂—(R_{C})—, —(R_{A})—NH(R_{B})—, —(R_{A})—N(R_{E})(R_{B})—, where R_{F}, R_{A} and R_{B} are optional, and, where present, R_{A}, R_{B}, R_{C}, R_{D} and R_{F} are individually chosen from alkanediyl, arenediyl, alkenediyl, alkynediyl, heteroarenediyl, cycloalkanediyl and heterocycloalkanediyl, and R_{E} is individually chosen from aryl, heteroaryl, alkyl, cycloalkyl, heterocycloalkyl, aralkyl, alkenyl and alkynyl.

It is preferred that the acridone derivative is linked to the solid support by an ester linkage. Such a linkage may typically be formed by providing an acridone derivative comprising a carboxyl group and a solid support comprising a hydroxyl group.

Referring to formula (I), it is preferred that the acridone derivative is linked to the solid support at the “5” position i.e. at the nitrogen atom in the acridone ring.

It is preferred that the acridone comprises an amino, mono- or di-substituted amino group, preferably attached to the acridone ring in the “2” position.

It is preferred that the solid support to which a fluorogenic reporter is attached has the general formula (III) below,

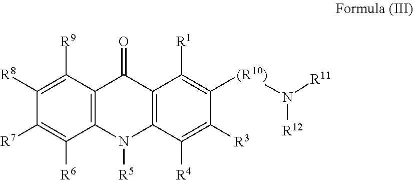

Wherein V is an optional linking group, S is a solid support, R¹⁰ is optionally present, and, if present, includes a hydrocarbon moiety that may comprise or include one or more moieties selected from aromatic hydrocarbon and aliphatic (straight or branched chain, saturated or unsaturated) moieties, each of which may be interrupted by and/or substituted or terminated by one or more substituents which may optionally include one or more heteroatoms,

and R¹¹ and R¹² are independently selected from H, a protecting group, an amino acid group or a peptide.

R¹⁰ may a include one or more of arenediyl, heteroarenediyl, alkanediyl, cycloalkanediyl, heterocycloalkanediyl, aralkanediyl, alkenediyl, alkynediyl, —(R_{A})—O—(R_{B})—, —(R_{A})—S—(R_{B})—, —R_{C}—R_{D}—, —(R_{A})—CO₂—(R_{B})—, —(R_{A})—O₂C—(R_{B}), —(R_{A})—CONH—(R_{B})—, —(R_{A})—NHC(O)—(R_{B})—, —(R_{A})—C(O)—(R_{C})—, —(R_{A})—OSO₂O—(R_{B})—, —(R_{A})—SO₂O—(R_{B})—, —(R_{A})—OSO₂—(R_{C})—, —(R_{A})—NH(R_{B})—, —(R_{A})—N(R_{E})(R_{B})—, where R_{F}, R_{A} and R_{B} are optional, and, where present, R_{A}, R_{B}, R_{C}, R_{D} and R_{F} are individually chosen from alkanediyl, arenediyl, alkenediyl, alkynediyl, heteroarenediyl, cycloalkanediyl and heterocycloalkanediyl, and R_{E} is chosen from aryl, heteroaryl, alkyl, cycloalkyl, heterocycloalkyl, aralkyl, alkenyl and alkynyl,

S is a solid support such as those typically used for the synthesis of peptides, for example, resins. The term “peptides” is taken to include polypeptides (proteins).

The linking group V, if present, may include a hydrocarbon moiety that may comprise or include one or more moieties selected from aromatic hydrocarbon and aliphatic (straight or branched chain, saturated or unsaturated) moieties, each of which may be interrupted by and/or substituted or terminated by one or more substituents which may optionally include one or more heteroatoms.

V preferably comprises one or more of arenediyl, heteroarenediyl, alkanediyl, cycloalkanediyl, heterocycloalkanediyl, aralkanediyl, alkenediyl, alkynediyl, —(R_{A})—O—(R_{B})—, —(R_{A})—S—(R_{B})—, —R_{C}—R_{D}—, —(R_{A})—CO₂—(R_{B})—, —(R_{A})—O₂C—(R_{B}), —(R_{A})—CONH—(R_{B})—, —(R_{A})—NHC(O)—(R_{B})—, —(R_{A})—C(O)—(R_{C})—, —(R_{A})—OSO₂O—(R_{B})—, —(R_{A})—SO₂O—(R_{B})—, —(R_{A})—OSO₂—(R_{C})—, —(R_{A})—NH(R_{B})—, —(R_{A})—N(R_{E})(R_{B})—, where R_{F}, R_{A} and R_{B} are optional, and, where present, R_{A}, R_{B}, R_{C}, R_{D} and R_{F} are individually chosen from alkanediyl, arenediyl, alkenediyl, alkynediyl, heteroarenediyl, cycloalkanediyl and heterocycloalkanediyl, and R_{E} is chosen from aryl, heteroaryl, alkyl, cycloalkyl, heterocycloalkyl, aralkyl, alkenyl and alkynyl. The linking group preferably comprises an ester linkage.

The protecting groups may be those that are commonly used to protect amino groups. Examples are described above in relation to the method of the first aspect of the present invention. Examples of these protecting groups are —(R_{AT}), —SO₂—(R_{AT}), —CO₂—(R_{AT}) and —Ph(CO)₂ [thus forming a phthalimide group] wherein R_{AT} is individually chosen from aryl, heteroaryl, alkyl, cycloalkyl, heterocycloalkyl, aralkyl, alkenyl, alkynyl and —R_{UT}—R_{UG} wherein R_{UT} is selected from alkanediyl, arenediyl, alkenediyl, alkynediyl, heteroarenediyl, cycloalkanediyl and heterocycloalkanediyl, and R_{UG} is chosen from aryl, heteroaryl, alkyl, cycloalkyl, heterocycloalkyl, aralkyl, alkenyl, alkynyl. Specific examples of the protecting groups have been described with reference to the first and second aspects of the present invention.

The amino acid or peptide group may be provided with one or more protecting groups, such as those that are well-known to those skilled in the art.

In accordance with the fourth aspect of the present invention, there is provided a kit for the synthesis of peptides, said kit comprising a solid support suitable for use in solid phase peptide synthesis, an acridone derivative optionally attached to the solid support or alternatively for reaction with the solid support to form an acridone derivative attached to the solid support, and compounds for the synthesis of peptides.

The acridone derivative may be as described in accordance with the method of the first aspect of the present invention. Alternatively, if the acridone derivative is attached to the solid support, then the solid support may be as described in accordance with the third aspect of the present invention.

In accordance with a fifth aspect of the present invention, there is provided the use of 2-aminoacridone or derivative provided with an amino group bonded to the “2” position (referring to Formula I) to label a peptide, the linkage between the 2-aminoacridone or derivative and the peptide being between said amino group and the peptide.

In accordance with an sixth aspect of the present invention there is provided a fluorogenic peptide as made in accordance with the method of the first aspect of the present invention. The structure of the peptide may be inferred from the method of the first invention. It is preferred that the fluorogenic peptide has the following structure:

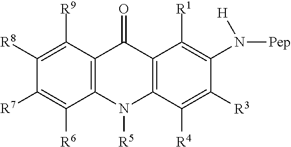

Where Pep is a peptide sequence, R⁵ comprises —CₙH₂ₙ-ACID, where n=1 to 12, wherein ACID is an acid group and R¹, R³, R⁴, R⁶, R⁷, R⁸ and R⁹ are independently selected from H and CₘH₂ₘ₊₁, where m=1 to 12.

Pep preferably comprises from 1 to 10 amino acid residues, more preferably from 4 to 10 amino acid residues. It is preferred that Pep comprises at least one of tyrosine, nitrotyrosine, nitrophenylalanine and tryptophan.

ACID preferably comprises carboxyl.

In accordance with a seventh aspect of the present invention, there is provided a method of determining the proteolytic activity of an enzyme suspected of being a proteolytic enzyme, the method comprising:
(a) providing a sample comprising a substrate to which is attached an acridone moiety, the acridone moiety being attached via a cleavage site for a proteolytic enzyme to a quenching moiety capable of quenching fluorescent radiation emitted by the acridone moiety
(b) providing the enzyme suspected of being a proteolytic enzyme in admixture with the cleavage site and
(c) illuminating the acridone moiety with radiation so as to cause the acridone moiety to fluoresce; and
(d) monitoring the properties of the light emitted from the sample.

Step (c) need not be performed after step (b); step (c) may be performed during one or both of steps (a) and (b).

The properties of the light emitted from the sample are characteristic of the environment of the acridone moiety, including the identity and proximity of the quenching moiety.

The quenching moiety is preferably provided by a peptide (in which case the substrate comprises a peptide). The quenching moiety is preferably an amino acid residue, more preferably tyrosine, tryptophan, nitrotyrosine, nitrophenylalanine or aspartic acid. When the quenching moiety is attached to the acridone moiety, the intensity of light emitted from the sample is lower than when the quenching moiety is liberated from the acridone moiety. Furthermore, when the quenching moiety is attached to the acridone moiety the decay time of the light emitted from the sample is shorter than when the quenching moiety is liberated from the acridone moiety.

More than one quenching moiety may be provided; for example, the substrate may be provided by a peptide which comprises more than one quenching moiety. This may be achieved using a peptide comprising more than one tyrosine, nitrotyrosine, nitrophenylalanine or tryptophan moiety.

The term “attached to” is not restricted to the case where the respective moieties or groups are directly attached to one another; there may be other species between the moieties or groups. For example, if the quenching moiety is a tyrosine residue, then there may be other amino acid residues between the acridone and the tyrosine moieties.

Step (d) may comprise monitoring the intensity of light as a function of time. The intensity of light may be the intensity of light at one particular wavelength, or may be an integrated intensity of light (this being the intensity summed over a range of wavelengths).

Step (d) may comprise measuring the decay time (sometimes called “lifetime”) as a function of time. The decay time may be indicative of the proximity of a quenching moiety to the acridone residue.

If the substrate comprises a peptide and the quenching moiety comprises an amino acid residue, in certain circumstances it is preferred that there is more than one amino acid residue between the acridone moiety and the quenching moiety.

It is preferred that the acridone moiety is attached to a terminus of a peptide, preferably the peptide C terminus. This may be achieved if the acridone is in aminoacridone moiety. This attachment may be performed using the method of the first aspect of the present invention. The method may further comprise providing a substance suspected of being an inhibitor of a proteolytic enzyme. This facilitates the assessment of the inhibiting power of the suspected inhibitor. An effective inhibitor would inhibit the activity of the enzyme and so the effect of the enzyme on the substrate will be mitigated, with a consequential effect on the fluorescence characteristics of the sample. In this way, the present method also provides a measurement of the effectiveness of an enzyme inhibitor.

It is preferred that the acridone is attached to the substrate via the acridone 2 position.

The peptide used in the present method may be made in accordance with the method of the first aspect of the present invention.

In accordance with a eighth aspect of the present invention, there is provided a method of determining the phosphorylating activity of an enzyme, the method comprising:
(a) providing a substrate to which is attached an acridone moiety, the acridone moiety being attached to a quenching moiety capable of quenching fluorescent radiation emitted by the acridone moiety
(b) providing reagents for the phosphorylation of the quenching moiety and an enzyme suspected of having phosphorylating activity in admixture with the quenching moiety and
(c) illuminating the acridone moiety with radiation so as to cause the acridone moiety to fluoresce; and
(d) measuring the properties of the light emitted from the sample.

When the quenching moiety (such as the amino acid tyrosine) is phosphorylated the fluorescence intensity and lifetime increases. Monitoring of the fluorescence intensity or fluorescence lifetime therefore offers a means of measuring the phosphorylating activity of an enzyme.

The quenching moiety is preferably provided by a peptide. The quenching moiety is preferably an amino acid residue, more preferably tyrosine, nitrotyrosine, tryptophan, nitrophenylalanine or aspartic acid.

The term “attached to” is not restricted to the case where the respective moieties or groups are directly attached to one another; there may be other species between the moieties or groups. For example, if the quenching moiety is a tyrosine residue, then there may be other amino acid residues between the acridone and the tyrosine moieties.

Step (d) may comprise monitoring the intensity of light as a function of time. The intensity of light may be the intensity of light at one particular wavelength, or may be an integrated intensity of light (this being the intensity summed over a range of wavelengths).

Step (d) may comprise measuring the decay time (sometimes called “lifetime”) as a function of time. The decay time may be indicative of the proximity of a quenching moiety to the acridone residue.

If the substrate comprises a peptide and the quenching moiety comprises an amino acid residue, in certain circumstances it is preferred that there is more than one amino acid residue between the acridone moiety and the quenching moiety.

It is preferred that the acridone is attached to the substrate via the acridone 2 position.

It is preferred that the acridone moiety is attached to a terminus of a peptide, preferably the peptide C terminus. This may be achieved if the acridone is in aminoacridone moiety. This attachment may be performed using the method of the first aspect of the present invention.

Biochemical assays using variable lifetime as an assay parameter are most robust when sample variation is largely due to pipetting errors, whereas assays using the intensity of a fixed lifetime as a parameter are most robust when sample variation is largely due to the presence of various fluorescent compounds.

In the methods of the seventh and eighth aspects of the present invention, it is preferred that the substrate comprises a peptide, and that the acridone moiety is attached to the peptide at the “2” position, as indicated below

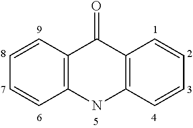

It is preferred that the acridone moiety is attached to the peptide via an amino link. It is preferred that the peptide comprises from 1 and 20 amino acid residues, preferably form 1 to 10, and more preferably from 4 to 10 amino acid residues. It is preferred that an acid group (preferably a carboxyl group) is attached to the “5” position of the acridone ring, preferably with a spacer group between the acid group and the nitrogen atom at the “5” position of the ring. The spacer group is preferably alkanediyl, preferably CₙH₂ₙ, where n is 1 to 12.

The invention is now described by way of example only by reference to the following figures of which:

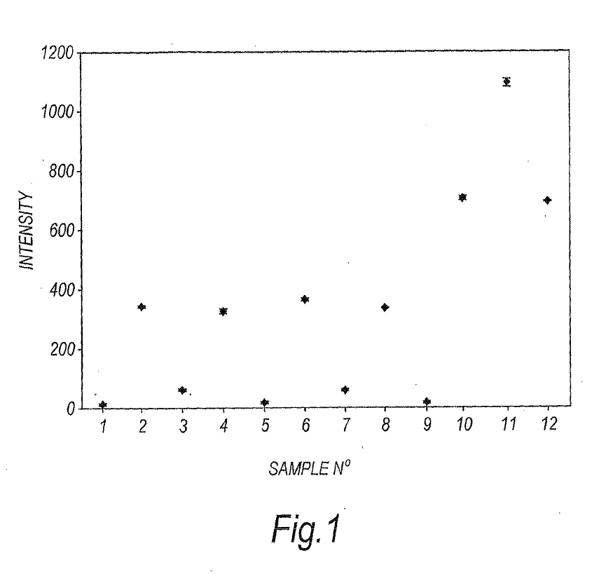

FIG. 1 shows the intensity of fluorescence for various samples associated with the method of the present invention;

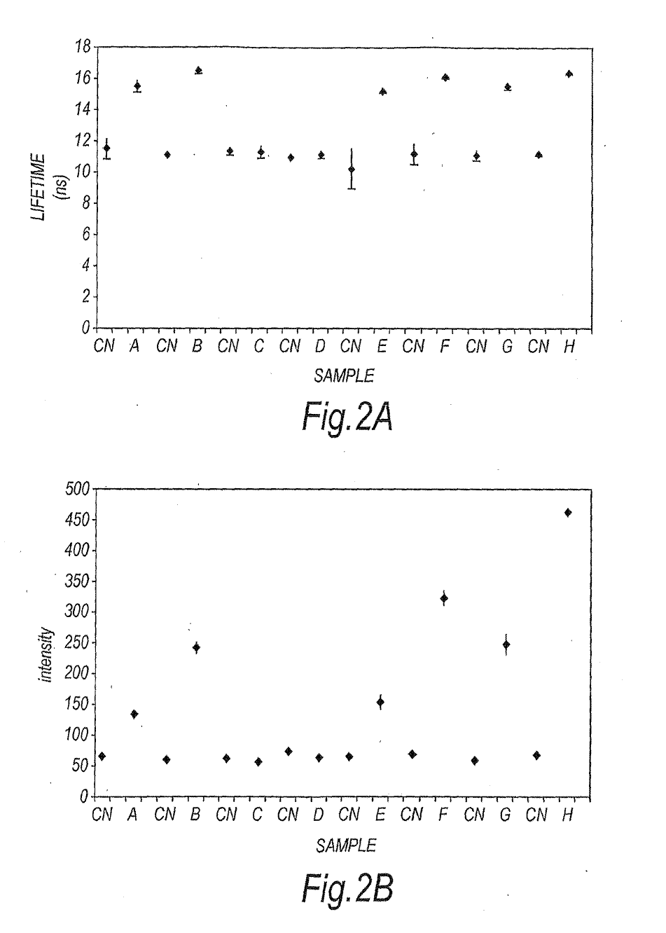

FIG. 2a shows the fluorescent lifetime (in ns) generated by various samples in admixture with various enzymes to illustrate a method of determining the proteolytic activity of enzymes, the fluorescence being measured in accordance with an example of the present invention;

FIG. 2b shows the fluorescent intensity generated by various samples in admixture with various enzymes to illustrate a method of determining the proteolytic activity of enzymes, the fluorescence being measured in accordance with an example of the present invention;

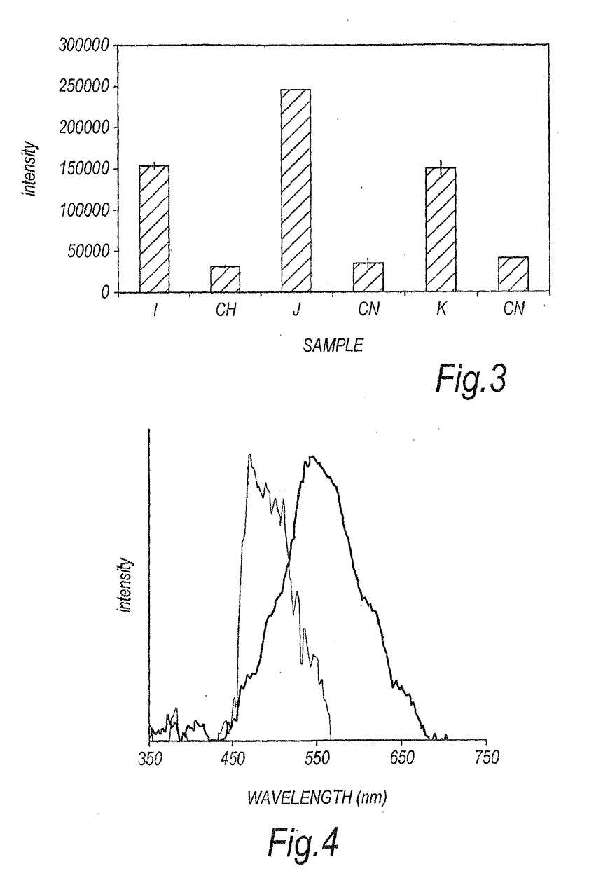

FIG. 3 shows the fluorescent intensity generated by various samples in admixture with various enzymes, the fluorescence being measured in accordance with an example of the present invention;

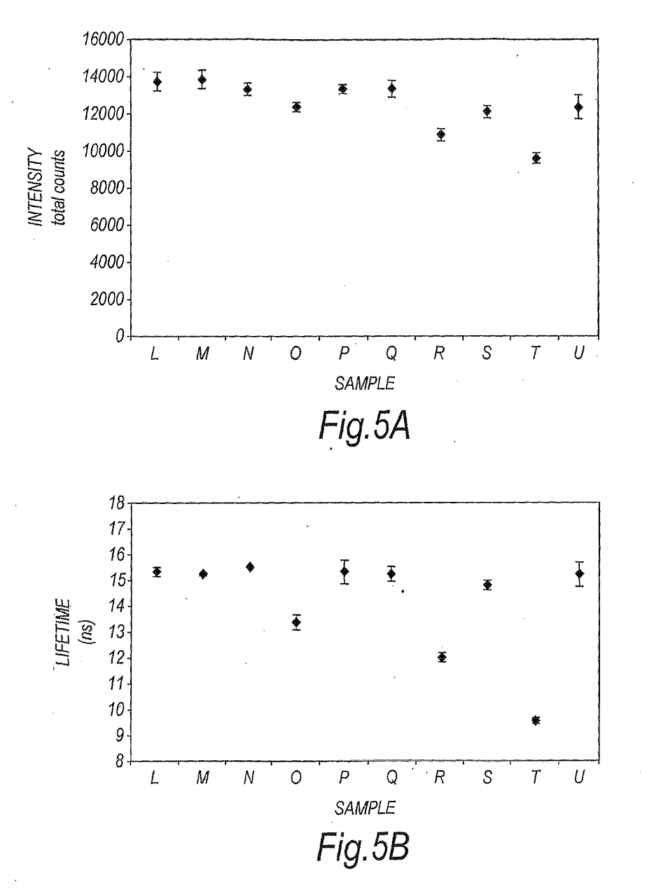

FIG. 4 shows the emission characteristics of a sample when the fluorophore is in proximity to (and remote from) a quencher;

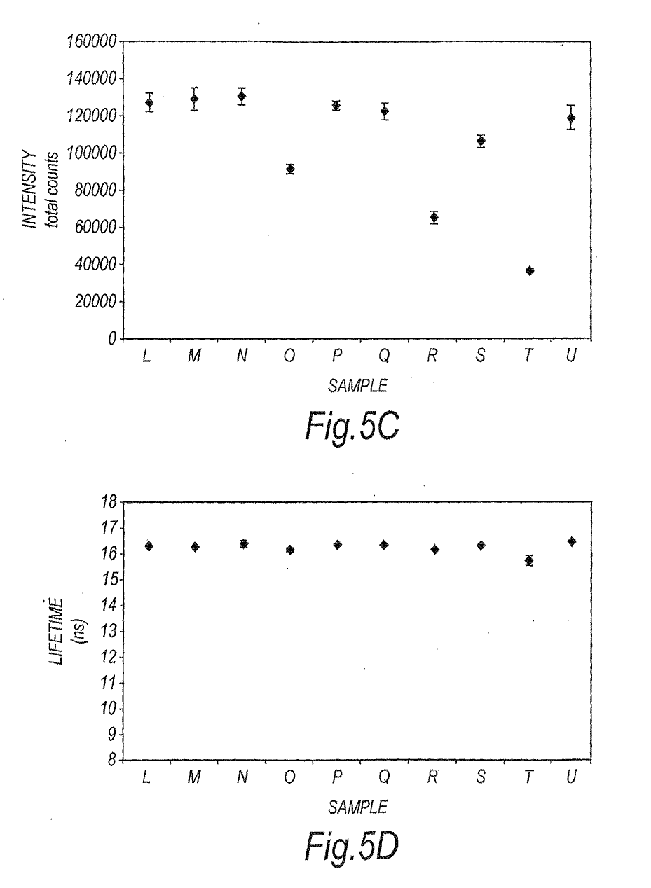

FIG. 5a shows the fluorescent intensity, measured at 555 nm, generated by various samples in admixture with various enzymes, the fluorescence being measured in accordance with an example of the present invention;

FIG. 5b shows the fluorescent lifetime, measured at 555 nm, generated by various samples in admixture with various enzymes, the fluorescence being measured in accordance with an example of the present invention;

FIG. 5c shows the fluorescent intensity, measured at 480 nm, generated by the samples of FIGS. 5a and 5b in admixture with various enzymes, the fluorescence being measured in accordance with an example of the present invention;

FIG. 5d shows the fluorescent lifetime, measured at 480 nm, generated by the samples of FIGS. 5a and 5b in admixture with various enzymes, the fluorescence being measured in accordance with an example of the present invention;

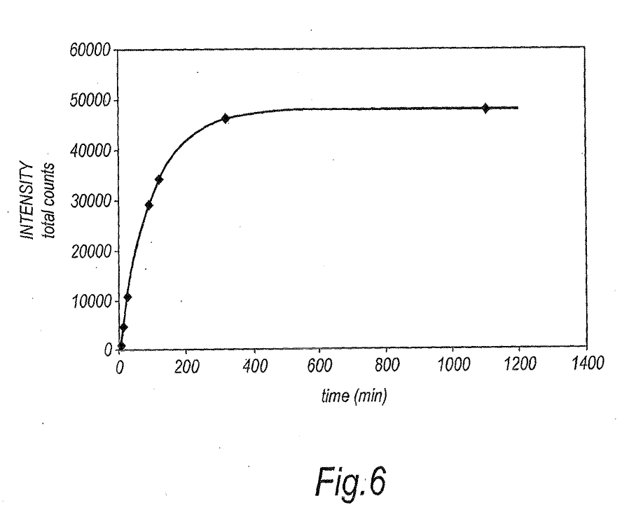

FIG. 6 shows the fluorescent intensity measured as a function of time after a proteolytic enzyme has been added to a substrate fluorogenic with an acridone moiety;

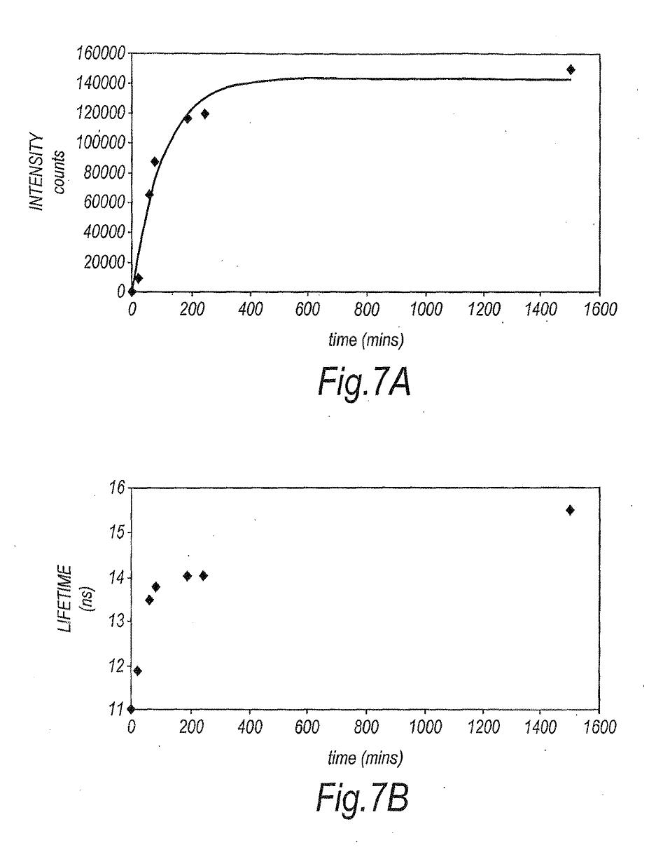

FIG. 7a shows the fluorescent intensity measured as a function of time when a peptide substrate is phosphorylated; and

FIG. 7b shows the fluorescent lifetime measured as a function of time when a peptide substrate is phosphorylated;

### EXAMPLE 1

Compounds of formula (IV), where n=1 or 5, were synthesised as described below.

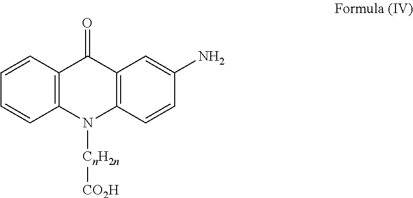

### Synthesis of 2-nitroacridone

Aniline (18.6 g, 200 mmol), 2-chloro-5-nitrobenzoic acid (20.1 g, 100 mmol), ethylene glycol (50 ml) and anhydrous sodium carbonate (10.6 g, 100 mmol) were placed in a reaction vessel and stirred until effervescence ceased. Cupric chloride (1.05 g, 7.5 mmol) dissolved in 20 ml of water was added to the reaction mixture. This was then heated to 125° C. for 6 hrs. The reaction was allowed to cool and water (300 ml) and charcoal were added. The mixture was filtered and then acidified to pH 2 with conc. hydrochloric acid. The precipitate was collected by filtration, washed with water and then re-dissolved in 1M sodium hydroxide solution. Material was re-precipitated by the addition of acetic acid, filtered off, washed with aqueous acetic acid, then water and finally dried under vacuum over phosphorous pentoxide to give 11.35 g, 44 mmol of N-(phenyl)-5-nitroanthranilic acid (44% yield).

The N-(phenyl)-5-nitroanthranilic acid (10 g, 39 mmol) and phosphorous oxychloride (40 ml) were stirred together and heated to 115° C. for 3.5 hours, then allowed to cool. The reaction mixture was placed on ice and small pieces of ice added, a vigorous reaction occurred with the evolution of hydrogen chloride. When the reaction had subsided, water (150 ml) was added and the mixture was boiled for 2 hours. On cooling, a solid precipitated out. This was filtered off and washed with water until the filtrate was colourless. The precipitate was further washed with cold methanol then diethyl ether and dried under vacuum to give 8.6 g, 36 mmol (92%) of 2-nitroacridone (92% yield).

The structure of 2-nitroacridone was confirmed by ¹H NMR. ¹H NMR (300 MHz) (d₆-DMSO) δ 12.2 (s, 1H), δ 8.92 (s, 1H), δ 8.4 (d, 1H), δ 8.2 (d, 1H), δ 7.8 (t, 1H), δ 7.6 (dd, 2H), δ 7.35 (t, 1H).

Two functional groups (an amino and an acid group) were incorporated into the acridone derivative as now described to form the compounds of Formula (IV).

### Synthesis of 6-(2-amino-9-oxo-9H-acridin-10-yl)-acetic acid (Formula IV, wherein n=1)

2-Nitroacridone (6 g, 25 mmol) was stirred with anhydrous dimethyl sulphoxide (65 ml) under a nitrogen atmosphere. After 5 minutes, sodium hydride (60% dispersed in oil, 1.2 g, 30 mmol) was added to the yellow solution. Stirring was continued for 90 mins. during which time the solution turned magenta. Ethyl 6-bromoacetate (3.3 ml, 30 mmol) was added and stirring continued overnight. The reaction mixture was poured into water (750 ml) and the yellow precipitate was collected by filtration, washed with water and dried under vacuum. The solid was dissolved in dichloromethane and anhydrous magnesium sulphate added to the solution. After filtration the solution was evaporated to dryness to leave a yellow-brown solid. The crude product was purified by flash chromatography (silica, 0-5% ethyl acetate:dichloromethane) to give 4.35 g, 13 mmol (53%) O-ethyl-6-(2-nitro-9-oxo-9H-acridin-10-yl)-acetate.

O-ethyl-6-(2-nitro-9-oxo-9H-acridin-10-yl)-acetate (4 g, 12 mmol), stannous chloride dihydrate (13.5 g, 60 mmol) and water (50 ml) were stirred for 2 hours at 40° C. under a nitrogen atmosphere.

The solution was cooled and the solvent removed by rotary evaporation. The residue was extracted with dichloromethane and filtered through celite. The dichloromethane solution was washed with saturated sodium bicarbonate solution and then water. The organic layer was dried with anhydrous magnesium sulphate, filtered and the solvent removed by rotary evaporation. The crude product was purified by flash chromatography (silica. 4-6% methanol:dichloromethane) to give 2.9 g, 9.6 mmol (80%) of O-ethyl-6-(2-amino-9-oxo-9H-acridin-10-yl)-acetate.

This was dissolved in ethanol (100 ml) and 1M sodium hydroxide (20 ml), and heated to 90° C. for 90 minutes. The solution was cooled, water added to give a yellow precipitate. The mixture was cooled in ice and acidified with hydrochloric acid when more material precipitated out. The precipitate was filtered off, washed with water and then ethanol and then dried under vacuum over phosphorous pentoxide to give 1.8 g, 6.9 mmol (72%) of 6-(2-amino-9-oxo-9H-acridin-10-yl)-acetic acid. The structure of the synthesized compound was confirmed by ¹H NMR. ¹H NMR (500 MHz) (d₆-DMSO) δ 8.3 (d, 1H), δ 7.7 (t, 1H), δ 7.56 (d, 1H), δ 7.5 (s, 1H), δ 7.45 (d, 1H), δ 7.2 (m, 2H), δ 5.25 (s, 2H), δ 2.5(s, 2H).

### Synthesis of 6-(2-amino-9-oxo-9H-acridin-10-yl)-hexanoic acid (Compound IV wherein n=5)

2-Nitroacridone (6 g, 25 mmol) was stirred with anhydrous dimethyl sulphoxide (65 ml) under a nitrogen atmosphere. After 5 minutes, sodium hydride (60% dispersed in oil, 1.2 g, (30 mmol) was added to the yellow solution. Stirring was continued for 90 mins during which time the solution turned magenta. Ethyl 6-bromohexanoate (5.33 ml, 30 mmol) was added and stirring continued overnight. The reaction mixture was poured into water (750 ml) and the yellow precipitate was collected by filtration, washed with water and dried under vacuum. The solid was dissolved in dichloromethane and anhydrous magnesium sulphate added to the solution. After filtration the solution was evaporated to dryness to leave a yellow-brown solid. The crude product was purified by flash chromatography (silica, 0-5% ethyl acetate:dichloromethane) to give 5.38 g, 14 mmol (56%) of O-ethyl-6-(2-nitro-9-oxo-9H-acridin-10-yl)-hexanoate.

O-ethyl-6-(2-nitro-9-oxo-9H-acridin-10-yl)-hexanoate (4.6 g, 12 mmol), stannous chloride dihydrate (13.5 g, 60 mmol) and water (50 ml) were stirred for 2 hours at 40° C. under a nitrogen atmosphere.

The solution was cooled and the solvent removed by rotary evaporation. The residue was extracted with dichloromethane and filtered through celite. The dichloromethane solution was washed with saturated sodium bicarbonate solution and then water. The organic layer was dried with anhydrous magnesium sulphate, filtered and the solvent removed by rotary evaporation. The crude product was purified by flash chromatography (silica. 4-6% methanol:dichloromethane) to give 3.8 g, 10.8 mmol (90%) of O-ethyl-6-(2-amino-9-oxo-9H-acridin-10-yl)-hexanoate.

This was dissolved in ethanol (100 ml) and 1M sodium hydroxide (20 ml), and heated to 90° C. for 90 minutes. The solution was cooled, water added to give a yellow precipitate. The mixture was cooled in ice and acidified with hydrochloric acid when more material precipitated out. The precipitate was filtered off, washed with water and then ethanol and then dried under vacuum over phosphorous pentoxide to give 2.64 g, 8.1 mmol (75%) of 6-(2-amino-9-oxo-9H-acridin-10-yl)-hexanoic acid. The structure of the synthesized compound was confirmed by ¹H NMR. ¹H NMR (500 MHz) (d₆-DMSO) δ 8.3 (d, 1H), δ 7.7 (m, 2H), δ 7.6 (d, 1H), δ 7.5 (s, 1H), δ 7.2 (m, 2H), δ 5.2 (broad s, 2H), δ 4.4 (t, 2H), δ 2.25(t, 2H), δ 1.8(m, 2H), 1.16 (m, 2H), δ 1.15 (m, 2H).

A protecting Fmoc group was then attached to the amino group to form compounds of formula (V), where n=1 or 5, as described below.

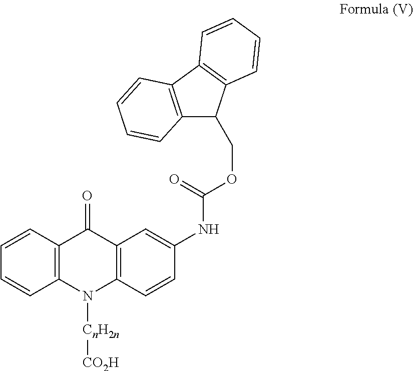

### Synthesis of 6-(2-(Fmoc-amino)-9-oxo-9H-acridin-10-yl)-acetic acid

4.97 g (19.2 mmol) 9-Fluorenylmethoxycarbonyl chloride (Fmoc chloride) was dissolved in 1,4-dioxane (25 ml) to give a clear solution. This was added dropwise to a solution of 6.56 g (20 mmol) of 6-(2-amino-9-oxo-9H-acridin-10-yl)-acetic acid and 2.76 g (20 mmol) potassium carbonate dissolved in dioxane/water (3:1) The solution was stirred at room temperature for 3-4 hours. The dioxane was removed under reduced pressure and the resulting oil was acidified with 10%v/v hydrochloric acid to give a pale yellow/brown precipitate. This was filtered off and washed with water and then dried in vacuo. to give 4.9 g (10 mmol) of 6-(2-(Fmoc-amino)-9-oxo-9H-acridin-10-yl)-acetic acid.

¹H NMR (300 MHz) (d₆-DMSO) δ 9.9 (s, 1H), δ 8.5 (s, 1H), δ 8.35 (d, 1H), δ 7.9 (d, 2H), δ 7.75 (d, 2H), δ 7.65 (t, 1H), δ 7.3-7.5 (m, 9H), δ 5.3(s, 2H), δ 4.55(d, 2H), δ 4.35 (t, 1H).

### Synthesis of 6-(2-(Fmoc-amino)-9-oxo-9H-acridin-10-yl)-hexanoic acid

4.97 g (19.2 mmol) 9-Fluorenylmethoxycarbonyl chloride (Fmoc chloride) was dissolved in 1,4-dioxane (25 ml) to give a clear solution. This was added dropwise to a solution of 7.68 g (20 mmol) of 6-(2-amino-9-oxo-9H-acridin-10-yl)-hexanoic acid and 2.76 g (20 mmol) potassium carbonate dissolved in dioxane/water (3:1) The solution was stirred at room temperature for 3-4 hours. The dioxane was removed under reduced pressure and the resulting oil was acidified with 10% v/v hydrochloric acid to give a pale yellow/brown precipitate. This was filtered off and washed with water and then dried in vacuo. to give 5.5 g (50 mmol) (50%) of 6-(2-(Fmoc-amino)-9-oxo-9H-acridin-10-yl)-hexanoic acid.

¹H NMR (300 MHz) (d₆-DMSO) δ 12.0 (s, 1H), δ 9.95 (s, 1H), δ 8.5 (s, 1H), δ 8.35 (d, 1H), δ 7.9 (d, 2H), δ 7.8 (d, 2H), δ 7.3-7.5 (m, 9H), δ4.3-4.6 (m, 6H), δ 2.25(t, 2H), δ 1.8 (m, 2H), δ 1.6 (m, 6H).

The compounds of formula (V) were then attached to a solid phase resin (a 2-chlorotrityl resin) by reacting the acid group with a chloro group provided on a resin. This attachment can be effected by using 4 equivalents of diisopropyldiethylamine (DIEA). All remaining chloro groups on the resin were then capped by reacting with methanol/DIEA.

This provides a solid support linked to a fluorogenic reporter with a protected amino moiety which can be used as the starting point for peptide synthesis.

### EXAMPLE 2

### Synthesis of Peptides

Peptides were synthesised using a SYRO (MultiSynTec Gmbh, Germany) peptide synthesiser using the Fmoc/BUT strategy. Couplings were performed using 3-6 equivalents Fmoc-amino acids/HOBt/TBTU and 6-12 equivalents N-methylmorpholine in order. TBTU is (N-[1H-benzotriazol-1-yl)(dimethylamino)methylene]-N-methylmethanaminium tetrafluoroborate N-oxide and HOBt is(1-Hydroxybenzotriazole).

The Fmoc groups were removed (i.e. the peptides were deprotected) and the peptides cleaved from the resins using trifluoroacetic acid (TFA)/water/triisopropylsilane (95/2.5/2.5%) for 2 hours at ambient temperature.

Cleaved peptides were purified by reverse phase HPLC on a Shimadzu LC-8A chromatograph with a SPD-6A uv-vis detector. The column used was an Ultrasep ES (RP-18), 250×20 mm column with 10 micron particle size. The eluent solvents used were: firstly, 0.05% TFA in water and secondly, 0.05% TFA in acetonitrile/water (80:20).

The purified peptides were analyzed by reverse phase HPLC on a Shimadzu LC-9A chromatograph with a SPD-M6A diode array detector. The column used was an Ultrasep ES (RP-18) 250×3 mm column with 7 micron particle size. The eluent solvents used were: firstly, 0.05% TFA in water and secondly, 0.05% TFA in acetonitrile/water (80:20).

The purified peptides were further characterised by MALDI-TOF mass spectrometry using a MALDI 2 DE mass spectrometry instrument (Shimadzo, Japan) in linear mode.

The mass spectrometry data were examined to ensure that they were consistent with the intended structure of the peptide

The following peptides were synthesised.

Peptide 1

Ac-WSDEVD-(2-aa-2)

The calculated molecular weight of 1042.8 compared well with the measured weight of 1043.6. The peptide was 95% pure. Fluorescent lifetime of 9.6 ns.

Peptide 2

Ac-WSDEVD-(2-aa-6)

The calculated molecular weight of 1098.8 compared well with the measured weight of 1099.8. The peptide was 95% pure. Fluorescent lifetime of 9.9 ns.

Peptide 3

Ac-SDEVD-(2-aa-2)

The calculated molecular weight of 856.3 compared well with the measured weight of 856.9. The peptide was 95% pure. Fluorescent lifetime of 16.0 ns.

Peptide 4

Ac-IETD-(2-aa-2)

The calculated molecular weight of 769.3 compared well with the measured weight of 770.6. The peptide was 95% pure. Fluorescent lifetime of 15.5 ns.

Peptide 5

Ac-YVAD-(2-aa-2)

The calculated molecular weight of 759.3 compared well with the measured weight of 760.1. The peptide was 95% pure. Fluorescent lifetime of 11.9 ns.

Peptide 6

Ac-LEHD-(2-aa-2)

The calculated molecular weight of 805.3 compared well with the measured weight of 806.2. The peptide was 95% pure. Fluorescent lifetime of 15.5 ns.

Peptide 7

4-NO₂-FSDEVD-(2-aa-2)

The calculated molecular weight of 1006.7 compared well with the measured weight of 1007.5. The peptide was 95% pure. Fluorescent lifetime of 9.5 ns.

Peptide 8

Ac-EPEGIYGVLF-(2-aa-2)

The calculated molecular weight of 1416.3 compared well with the measured weight of 1438.8. The peptide was 95% pure. Fluorescent lifetime of 10.6 ns.

Peptide 9

Ac-EPEGIYGVLF-(2-aa-6)

The calculated molecular weight of 1472.3 compared well with the measured weight of 1473.1. The peptide was 95% pure. Fluorescent lifetime of 10.6 ns.

In the structures above, Ac is acetyl (indicating that the attached residue is an acetylated residue), 2-aa-2 is 6-(2-amino-9-oxo-9H-acridin-10-yl)-acetic acid, 2-aa-6 is 6-(2-amino-9-oxo-9H-acridin-10-yl)-hexanoic acid and 4-NO₂—F is 4-nitrophenylalanine.

The fluorescence lifetimes of the synthesised peptides recorded above were measured by time correlated single photon counting. Peptides were dissolved in aqueous buffer solutions and excited at 405 nm using a pulsed solid state laser diode (Picoquant LDH-405). Fluorescent emission was filtered through a narrow bandpass interference filter (480/20 nm) and single photons were detected by a photomultiplier tube detector (Hamamatsu R7400P) and counted using a fast timing board with histogramming memory (Ortec 9353). All peptides were found to be fluorescent. When the amino acids tyrosine, tryptophan or nitro-phenylalanine (Y, W, NO₂—F) are included in the fluorogenic peptide, the fluorescence lifetime of the peptide is reduced (quenched) by 5-6 ns. This is useful because they can be inserted into an amino acid sequence to serve as indicators of the integrity of the peptide.

The present invention relates to the manufacture of fluorogenic peptides. The acridone reporter is fluorescent, and the properties of the fluorescence reflect the environment of the acridone reporter, including the identity and proximity of certain amino acid groups in the peptide. For example, certain amino acids, in particular, tyrosine, nitrotyrosine, nitrophenylalanine and tryptophan can quench the fluorescence of acridone moieties. This can be seen as a decrease in fluorescence lifetime and intensity. For example, the fluorescence lifetime of adducts of 2-aminoacridone attached directly to the α-carboxylic acid group of N-acetyl-L-alanine, N-acetyl-L-tyrosine and N-acetyl-L-tryptophan are 18.1, 9.4 and 3.0 nanoseconds respectively. The structures of these derivatives are shown in formula VIII.

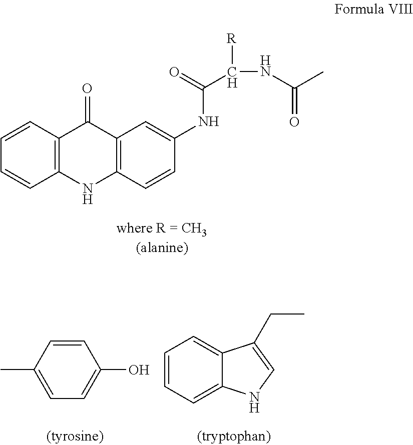

Synthesis of a peptide containing a tyrosine, nitrotyrosine, nitrophenylalanine or tryptophan residue in proximity to a 2-aminoacridone moiety attached to a solid phase as outlined above result in products with a shortened fluorescence lifetime compared to similar peptides which do not contain either of these amino acids. The degree of lifetime shortening depends on the position of the tyrosine or tryptophan residue relative to the 2-aminoacridone moiety. The shorter the distance, the greater the degree of quenching and the shorter the fluorescence lifetime.

### EXAMPLE 3

FIG. 1 shows how the proximity of certain amino acid moieties to the 2-aminoacridone moiety affects the fluorescence characteristics of the reaction mixture.

Peptides 1 and 2 were mixed with caspase 3 enzyme in order to demonstrate the enzyme activity of caspase 3. Caspase 3 cleaves WSDEVD-(2aa2) and WSDEVD-(2aa6) at the second aspartic acid residue. The composition of the samples shown in FIG. 1 are as follows:

| Sample No. | Description of Sample |
|---|---|
| 1, 5, 9 | Buffer |
| 2 | Peptide 1 |
| 3 | Peptide 1 + caspase 3 |
| 4 | Peptide 1 + caspase 3 + zinc |
| 6 | Peptide 2 |
| 7 | Peptide 2 + caspase 3 |
| 8 | Peptide 2 + caspase 3 + zinc |
| 10 | PT14 peptide |
| 11 | PT14 + caspase 3 |
| 12 | PT14 + caspase 3 + zinc |

PT14 peptide is WSDEVD-PT14, a conventionally labeled peptide, where PT14 is the Puretime 14 moiety. PT14 peptide is also known as peptide 10, but is not made in accordance with the method of the present invention. Puretime 14 is available from AssayMetrics Limited, Cardiff, UK.

As can be seen from FIG. 1, there is a large change in signal intensity (83% change) when the (2aa2) and (2aa6) moieties are cleaved by the caspase 3 enzyme. Such a change in intensity was unexpectedly high and compared very favourably with the 55% change in intensity when the Puretime 14 moiety was cleaved from the peptide (samples 10 and 11). As expected, the presence of zinc ions (1 mM Zn²⁺) inhibited the action of the enzyme.

### EXAMPLE 4

Use can be made of this phenomenon of the change in fluorescence properties in several biological applications. For example, if the peptide contains a cleavage site for proteolytic enzyme between the tyrosine/tryptophan residue and the aminoacridone moiety, then cleavage of the peptide results in the removal of the quenching residue. This leads to an increase in fluorescence lifetime. For example the fluorogenic peptide N-acetyl-ileu-tyr-gly-glu-phe with 2-aminoacridone on the C-terminal phe residue has a short fluorescence lifetime. Digestion with a proteolytic enzyme causes the fluorescence lifetime to increase.

### EXAMPLE 5

A further use of the change in fluorescence properties described with reference to Example 3 involves tyrosine-containing peptides. Certain tyrosine containing peptides can act as tyrosine kinase substrates. If 2-aminoacridone is bonded to a peptide containing tyrosine and the tyrosine is subsequently phosphorylated it will show an increase in fluorescence lifetime.

### EXAMPLE 6

This example illustrates how measurement of the fluorescent lifetime and the measurement of fluorescent intensity can be used to monitor the activity of four enzymes (pepsin, asp-N endopeptidase, thermolysin and chymotrypsin) towards two of the peptides (numbers 8 and 9) mentioned above.

The enzyme activity of each of the four proteases towards the fluorogenic peptides 8 and 9 was detected by measuring changes in fluorescence intensity and lifetime. Changes resulted from cleavage of the peptides so that the quenching tyrosine residue was separated from the (2-aa-2) fluorophor. All enzymes reactions were performed in black polypropylene 384 well plates (Matrix Technologies Ltd), with a final assay volume of 100 microlitres of aqueous buffer. Assays were formatted in triplicates with no-enzyme controls for each enzyme:

i) The pepsin assay was performed in citrate buffer (0.1M/pH 3.6) containing substrate peptides (500 nM) and pepsin enzyme (˜1 ng per well). Reaction was initiated by addition of the enzyme.

ii) The asp-N assay was performed in Tris buffer (0.1M/pH 8.5) containing substrate peptides (500 nM) and asp-N enzyme (˜1 ng per well). Reaction was initiated by addition of the enzyme.

iii) The chymotrypsin assay was performed in Tris buffer (0.05M/pH 7.6) containing CaCl₂ (0.01M), substrate peptides (500 nM) and chymotrypsin enzyme (˜1 ng per well). Reaction was initiated by addition of enzyme.

iv) The thermolysin assay was performed in PBS buffer (0.1M/pH 7.4) containing CaCl₂ (0.01M), substrate peptides (500 nM) and thermolysin enzyme (˜1 ng per well). Reaction was initiated by addition of enzyme.

Fluorescence emission from all four sets of assay wells was measured at 450 nm as indicated above, after incubation at room temperature (20° C.) for four hours.

FIGS. 2a and 2b demonstrate the action of each of the four enzymes on the two substrates and shows pronounced changes in fluorescence lifetime upon cleavage.

The composition of the samples used to generate the data shown in FIGS. 2a and 2b are as follows:

| Sample | Composition |
|---|---|
| A | Peptide 9 + pepsin |
| B | Peptide 8 + pepsin |
| C | Peptide 9 + aspn |
| D | Peptide 8 + aspn |
| E | Peptide 9 + chymo |
| F | Peptide 8 + chymo |
| G | Peptide 9 + thermo |
| H | Peptide 8 + thermo |

“Pepsin” is pepsin enzyme, “aspn” is asp-N endopeptidase. “Thermo” is thermolysin and “chymo” is chymotrypsin.

All samples labelled CN are no-enzyme controls. The result for the appropriate control for a particular sample is immediately to the left of the result for that sample.

Peptides 8 and 9 show large changes in fluorescence lifetime and intensity when incubated with pepsin, chymotrypsin and thermolysin, but no change when incubated with asp-N. The large changes are associated with the cleavage of the tyrosine quencher from the acridone moiety.

FIG. 3 illustrates how enzyme activity can be detected by the cleavage of nitrophenylalanine and tryptophan moeities.

Caspase 1 and asp-N enzymes were used to demonstrate the effectiveness of fluorogenic peptides containing all three quenching species.

These enzymes reactions were performed in black polypropylene 384 well plates (Matrix Technologies Ltd), with a final assay volume of 100 microlitres of aqueous buffer. Assays were formatted in triplicates with no-enzyme controls for each enzyme:

i) The caspase 1 assay was performed in 0.1M phosphate buffer containing DTT (0.01M), peptide Ac-YVAD-(2-aa-2) (100 μM), caspase 1 enzyme (0.5 unit per well). Reaction was initiated by addition of the enzyme.

ii) The asp-N assays were performed in Tris buffer (0.1M/pH 8.5) containing substrate peptides 4-NO₂—FSDEVD-(2-aa-2) (500 nM) or Ac-WSDEVD-(2-aa-2) (500 nM) and asp-N enzyme (˜1 ng per well). Reaction was initiated by addition of the enzyme.

The composition of the samples used to generate the data shown in FIG. 3 is as follows:

| Sample | Composition |
|---|---|
| I | Peptide 5 + caspase 1 |
| J | Peptide 7 + aspn |
| K | Peptide 1 + aspn |

“Caspase 1” is caspase 1 enzyme and “aspn” is asp-N endopeptidase.

All samples labelled “CN” are no-enzyme controls. The result for the appropriate control for a particular sample is immediately to the right of the result for that sample.

In the absence of an enzyme (“CN”) that cleaves the peptide, small signals are observed but when enzyme activity cleaves the peptides, a large signal is obtained.

Fluorescent lifetime is measured by monitoring the decay time of the fluorescence signal.

FIG. 4 shows the emission spectra obtained in aqueous Tris buffer of the substrate fluorogenic peptide 5 (the more faint line) and the cleavage product 2-aa-2 (2-aminoacridone-N-ethanoic acid), the heavier line denoting the spectrum of the cleavage product. The peak wavelength of the emitted fluorescence shifts towards the red region of the spectrum when a fluorogenic peptide is cleaved specifically at the carbon terminus.

Not only does the wavelength of the fluorescence shift when the peptide sequence is cleaved from the ring, but the fluorescence lifetime also changes. The lifetime can be made to change to a large or small degree by the insertion of appropriate amino acids into the peptide sequence. For example, 2-aa-2 has a fluorescence lifetime of around 10 ns while the peptide 3 has a lifetime of 16 ns. When the quenching amino acid W (tryptophan) is added to the N terminus of the peptide, the peptide 1 is found to have a lifetime of only 9.6 ns. The extendable nature of the modified (2-aa-2) fluorophor therefore makes it possible to design peptide substrates that have a characteristic long fluorescence lifetime that changes upon enzyme activity or a characteristic long fluorescence lifetime that does not change upon enzyme activity.

FIGS. 5a, 5b, 5c and 5d shows how intensity measurements and lifetime measurements may be used to monitor the effect of certain enzyme inhibitors on the action of enzymes.

The composition of the samples used to generate the data shown in FIGS. 5a, 5b, 5c and 5d is as follows:

| Sample | Composition |
|---|---|
| L | Peptide 3 + caspase 3 + inhibitor 1 |
| M | Peptide 3 + caspase 3 + inhibitor 2 |
| N | Peptide 3 + caspase 3 + inhibitor 3 |
| O | Peptide 3 + caspase 3 + inhibitor 4 |
| P | Peptide 3 + caspase 3 + inhibitor 5 |
| Q | Peptide 3 + caspase 3 + inhibitor 6 |
| R | Peptide 3 + caspase 3 + inhibitor 7 |
| S | Peptide 3 + caspase 3 + inhibitor 8 |
| T | Peptide 3 + caspase 3 (positive control) |
| U | Peptide 3 (negative control) |

All of the inhibitors were from the Caspase Inhibitor Set II (Catalogue no. 218772) from the Calbiochem range supplied by EMD Chemicals, Inc., San Diego, Calif., USA.

Inhibitor 1 is Caspase-1 Inhibitor VI, Z-YVAD-FMK (Cat. No. 218746). Inhibitor 2 is Caspase-2 Inhibitor I, Z-VDVAD-FMK (Cat. No. 218744). Inhibitor 3 is Caspase-3 Inhibitor II, Z-DEVD-FMK (Cat. No. 264155). Inhibitor 4 is Caspase-5 Inhibitor I, ZWEHD-FMK (Cat. No. 218753). Inhibitor 5 is Caspase-6 Inhibitor I, Z-VEIDFMK (Cat. No. 218757). Inhibitor 6 is Caspase-8 Inhibitor II, Z-IETD-FMK (Cat. No. 218759). Inhibitor 7 is Caspase-9 Inhibitor I, Z-LEHD-FMK (Cat. No. 218761) and Inhibitor 8 is Caspase Inhibitor III, Boc-D-FMK (Cat. No. 218745).

Profiling of eight caspase inhibitors was performed in black polypropylene 384 well plates (Matrix Technologies Ltd), with a final assay volume of 100 microlitres of aqueous buffer. Assays were formatted in triplicates with a positive control containing no test inhibitor and a negative control containing no caspase 3 enzyme. Assays were performed in 0.1M phosphate buffer containing DTT (0.01M), peptide Ac-SDEVD-(2-aa-2) (10 μM), caspase 3 enzyme (˜20 ng per well) and caspase inhibitors 1, 2, 3, 4, 5, 6, 7 and 8 at concentrations of 1 ∥M. Reactions were initiated by addition of the enzyme. The assay plate was read using the fluorescence lifetime instrumentation described previously with blue (480 nm) and green (555 nm) bandpass filters.

Results of the experiment are shown in FIG. 5. In the absence of inhibitor, caspase 3 cleaves the peptide to the right of the aspartic acid (D), producing a shift in the fluorescence emission towards longer wavelength, resulting in a decrease in intensity measured at 480 nm (see FIG. 5c). When the fluorescence of the peptide is measured at 480 nm, the assay shows essentially no change in fluorescence lifetime.

However, when the fluorescence of the peptide is measured at 555 nm, the assay shows smaller changes in intensity but a very noticeable decrease in lifetime when the caspase 3 cleaves the peptide.

Therefore, monitoring the intensity at 480 nm is a good way of monitoring the activity of the enzyme. Likewise, monitoring the lifetime at 550 nm is a good way of monitoring the activity of the enzyme.

Biochemical assays using variable lifetime as an assay parameter are most robust when sample variation is largely due to pipetting errors, whereas assays using the intensity of a fixed lifetime as a parameter are most robust when sample variation is largely due to the presence of various fluorescent compounds. Biochemical assays performed with fluorogenic peptides according to the present invention will therefore be particularly advantageous in high throughput screening (where fluorescent compounds are frequently a problem) and compound profiling applications (where quantitation is important and frequently affected by pipetting errors).

This is because the fluorogenic peptides in the present invention permit modification of the degree of quenching by inclusion of an appropriate amino acid moeity and selection of a fluorescence detection wavelength that changes the measured fluorescence lifetime.

FIG. 6 shows that quantitative measurement of enzyme activity is possible by monitoring the evolution of the fluorescence signal over time. FIG. 6 shows the increase in fluorescence intensity that occurred when Peptide 2 was cleaved by the asp-N endoprotease. Fluorescence was measured at 450 nm using the instrumentation described previously. The reaction was carried out in a total volume of 20 μL of 0.2 M/7.4 pH tris buffer solution with the substrate WSDEVD-2-aa-6 initially at 500 nM concentration. Reaction was initiated by addition of the enzyme asp-N (final concentration 0.02 nM). Incubation was at 37° C. in a black low-binding low-volume 384 well microtitre plate (Corning 3676).

With Peptide 2 at excess, first order reaction kinetics are expected. The software Enzfitter (Biosoft, Cambridge UK) was used to fit the first order rate equation:

Total counts=offset+limiting counts×(1−exp(−k.t))

Where k is the first order rate constant and t is the time. k was found to be 1.036×10⁻² per min and the goodness of fit indicator X² was 0.1.

FIGS. 7a and 7b show that the fluorogenic peptides made by the method of the present invention are also effective substrates for phosphorylation by kinases.

The kinase assay was carried out in triplicate in a 384 well microtitre plate (Matrix Technologies #4308) using 50 mM Tris buffer (pH 7.2), 10 mM MgCl₂, Peptide 9 (600 nM) and ATP (40 μM). Reactions were initiated by the addition of 10 milli-units of enzyme in buffer solution to give final volumes of 100 μl. Fluorescence intensity and lifetime were measured at 450 nm using the equipment described previously, over a period of 1500 minutes.

FIG. 7a shows the increase in fluorescence intensity that occurred when Peptide 9 was phosphorylated. The software Enzfitter (Biosoft, Cambridge UK) was used to fit the first order rate equation to the fluorescence intensity (total counts):

Total counts=limiting counts×(1−exp(−k.t))

Where k is the first order rate constant and t is the time. k was found to be 9.91×10⁻³ per min.

FIG. 7b shows the increase in fluorescence lifetime that occurred when Peptide 9 was phosphorylated.

When the quenching amino acid tyrosine is phosphorylated by a kinase enzyme the fluorescence intensity and lifetime increases. Monitoring of the fluorescence intensity or fluorescence lifetime therefore offers a means of measuring the activity of a kinase enzyme.

1. A method of making a fluorogenic peptide with an acridone fluorophor, the method comprising the steps of:
(a) providing an acridone derivative, said acridone derivative having first and second reactive groups (or precursors thereof),
(b) providing a solid phase support provided with reactive species for reacting with the first reactive group of the acridone derivative
(c) causing the solid phase support and acridone derivative to react so that the acridone derivative is attached to the solid phase support
(d) providing the peptide or reagents for the formation of the peptide, and
(e) subsequent to step (c), causing the reaction of the second acridone reactive group with the peptide or one or more of the reagents for the formation of the peptide.

2. A method according to claim 1 wherein acridone derivative provided in step (a) includes or comprises a precursor of the first or second reactive group, and wherein the method further comprises treating the acridone derivative provided in step (a) such that the first or second reactive group is formed from the precursor.

3. A method according to claim 1 or claim 2 wherein the first reactive group comprises a carboxyl group.

4. A method according to claim 1 wherein the second reactive group comprises an amino group.

5. A method according to claim 2 wherein the precursor comprises a protecting moiety.

6. (canceled)

7. A method according to claim 1 wherein one or both of the first and second reactive groups (or precursors thereof) are linked to the acridone ring by a spacer group.

8. A method according to claim 1 wherein the second reactive group (or precursor thereof) is attached to the “2” or “4” position of the acridone, and the first reactive group (or precursor thereof) is attached to the “5” position of the acridone ring, the substituent positions being given below in Formula (1):

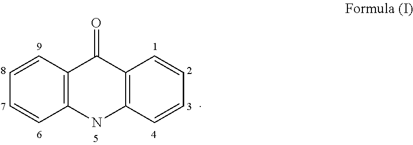

9. A method according to claim 8 wherein the first reactive group comprises a carboxyl group and the second reactive group comprises an amino group.

10. (canceled)

11. A method according to claim 1 wherein the one or more reagents for the formation of a peptide comprises at least one amino acid residue and an amino acid protecting group.

12. A method according to claim 11 comprising causing the at least one amino acid residue to react with the second reactive group.

13. A method according to claim 13 further comprising removing the amino acid protecting group and reacting a further amino acid residue with the deprotected amino acid residue.

14. A method according to claim 1 comprising the step of cleaving the peptide from the support.

15. A method according to claim 1 wherein the peptide comprises from 1 to 10 residues.

16. A method according to claim 1 wherein the peptide comprises at least one residue of one or more of tyrosine, nitrotyrosine, nitrophenylalanine and tryptophan.

17. A solid support to which a fluorogenic reporter is attached, the fluorogenic reporter being an acridone derivative.

18. A solid support to which a fluorogenic reporter is attached as claimed in claim 17 wherein the acridone is linked to the solid support via a linking group.

19. (canceled)

20. (canceled)

21. A kit for the synthesis of peptides, said kit comprising a solid support suitable for use in solid phase peptide synthesis, an acridone derivative optionally attached to the solid support or alternatively for reaction with the solid support to form an acridone derivative attached to the solid support, and compounds for the synthesis of peptides.

22. The use of 2-aminoacridone or derivative provided with an amino group attached to the “2” position (referring to Formula I) to form a fluorogenic peptide, the linkage between the 2-aminoacridone or derivative and the peptide being between said amino group and the peptide

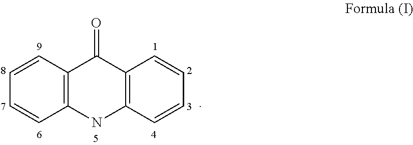

23. (canceled)

24. A fluorogenic peptide having the structure: 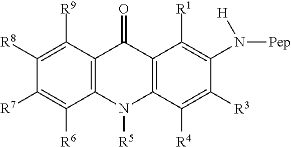
Where Pep is a peptide sequence, R⁵ comprises —CₙH₂ₙ-ACID, where n=1 to 12, wherein ACID is an acid group and R¹, R³, R⁴, R⁶, R⁷, R⁸ and R⁹ are independently selected from H and CₘH₂ₘ₊₁, where m=1 to 12.

25. A method of determining the proteolytic activity of an enzyme suspected of being a proteolytic enzyme, the method comprising:
(a) providing a sample comprising a substrate to which is attached an acridone moiety, the acridone moiety being attached via a cleavage site for a proteolytic enzyme to a quenching moiety capable of quenching fluorescent radiation emitted by the acridone moiety
(b) providing the enzyme suspected of being a proteolytic enzyme in admixture with the cleavage site and
(c) illuminating the acridone moiety with radiation so as to cause the acridone moiety to fluoresce; and
(d) monitoring the properties of the light emitted from the sample.

26. (canceled)

27. (canceled)

28. (canceled)
